(19) United States
(12) Patent Application Publication
(10) Pub. No.: US 2021/0161902 A1
(43) Pub. Date: Jun. 3, 2021

(54) EPICHAPEROME INHIBITOR THERAPY FOR TRAUMATIC BRAIN INJURY AND SEQUELAE THEREOF

(71) Applicant: Samus Therapeutics, Inc, Topsfield, MA (US)

(72) Inventor: Barbara P. Wallner, Cohasset, MA (US)

(73) Assignee: Samus Therapeutics, Inc., Topsfield, MA (US)

(21) Appl. No.: 16/624,274

(22) PCT Filed: Jun. 22, 2018
(86) PCT No.: PCT/US2018/038893

§ 371 Date: Dec. 18, 2019

Related U.S. Application Data
(60) Provisional application No. 62/524,452, filed on Jun. 23, 2017.
(60) Provisional application No. 62/532,989, filed on Jul. 14, 2017.

(51) Int. Cl. A61K 31/52; A61K 9/00; A61K 45/06; A61P 25/00

(52) U.S. Cl. CPC A61K 31/52; A61K 9/0053; A61K 9/0019; A61K 9/0043; A61K 9/0056; A61K 9/0073; A61K 45/06; A61P 25/00; A61K 9/0078

(57) ABSTRACT

Provided herein are methods of using certain Hsp90 inhibitors in treating subjects who have experienced or are experiencing a traumatic brain injury, including lessening the short term impact of such TBI and/or reducing the risk of developing and/or the severity of long term after effects of such TBI.

### RELATED APPLICATIONS

This application claims the benefit under 35 U.S.C. § 119(e) of U.S. Provisional Application Ser. No. 62/524,452, filed on Jun. 23, 2017 and U.S. Provisional Application Ser. No. 62/532,989, filed on Jul. 14, 2017, the entire contents of each of which are incorporated herein by reference.

### BACKGROUND

Hsp90, a heat shock protein, exists in an uncomplexed or weakly complexed state in normal cells called the chaperome which comprises Hsp90, co-chaperones and cellular proteins. In certain diseases, Hsp90 is complexed with aberrant proteins to form multi-component complexes and networks termed epichaperomes. Hsp90 is believed to act as a nucleating site for such complexes. The epichaperome components are physically and functionally integrated, and the epichaperome itself is proposed to enhance cellular survival, particularly of certain cells in diseases. Based on these various functions, the epichaperome has been identified as a target for certain therapies, including cancer and neurodegenerative disease therapies.

### SUMMARY

This disclosure is premised in part on the unexpected finding that certain inhibitors of Hsp90, Hsp90 isoforms and Hsp90 homologs that are able to cross the blood brain barrier (BBB) are useful in the treatment of traumatic brain injury (TBI) and in the prevention of long-term sequelae of TBI such as but not limited to chronic traumatic encephalopathy (CTE). These inhibitors are referred to herein as Hsp90 inhibitors or epichaperome inhibitors. They are able to bind selectively to Hsp90 including Hsp90 isoforms and homologs when these proteins are complexed in an epichaperome. Hsp90 inhibitors (or epichaperome inhibitors) that are able to cross the blood-brain barrier (BBB) are referred to as blood-brain barrier (BBB) permeable or BBB-permeable Hsp90 inhibitors or epichaperome inhibitors. The inhibitors of this disclosure provide therapeutic benefit, including prophylactic benefit, to subjects who have experienced one or repeated TBI. In some instances, early and optionally repeated use of such inhibitors can decrease the severity of acute TBI, reduce the risk of developing, delay the onset of, and/or reduce the severity of TBI sequelae such as but not limited to chronic traumatic encephalopathy (CTE).

Thus, in one aspect, provided herein is a method for treating a subject that has experienced traumatic brain injury (TBI) comprising administering to the subject an effective amount of a BBB-permeable epichaperome inhibitor anywhere from 1 hour to 6 months after the occurrence of the TBI.

The BBB-permeable epichaperome inhibitor may be administered within 5, 4, 3, 2 months or 1 month of the TBI. The BBB-permeable epichaperome inhibitor may be administered within 4, 3, 2 weeks or 1 week of the TBI. The BBB-permeable epichaperome inhibitor may be administered within 2 weeks of the TBI. The BBB-permeable epichaperome inhibitor may be administered within 10, 9, 8, 7, 6, 5, 4, 3, 2 days or 1 day of the TBI. The BBB-permeable epichaperome inhibitor may be administered within 24, 20, 16, 12, 8, 4, 3 or 2 hours, or 1 hour of the TBI. The BBB-permeable epichaperome inhibitor may be administered between (and including) 1 hour to 5 days after the TBI.

The BBB-permeable epichaperome inhibitor may be administered once or more than once (repeatedly). The BBB-permeable epichaperome inhibitor may be administered one or more times a day for a number of days, or one or more times a week for a number of weeks. For example, the BBB-permeable epichaperome inhibitor may be administered twice a day, three times a day, or four times a day, for 1 day or more. The frequency and duration of the treatment regimen may depend on the severity of the injury or when symptoms appear and/or the degree of inflammation experienced by the subject.

The subject may have experienced a concussive TBI (i.e., the subject has experienced a concussion).

The subject that has experienced a TBI will typically manifest one or more of the following symptoms including but not limited to headache or sensation of pressure in the head, temporary loss of consciousness, confusion, amnesia surrounding the traumatic event giving rise to the TBI, dizziness, ringing in the ears, nausea, vomiting, slurred speech, delayed responsiveness (e.g., delayed response to questions), appearing dazed, fatigue, pupil dilation, compromised vision, and difficulty breathing. One or more symptoms may arise immediately after the traumatic event, or they may arise within hours or even days of the traumatic event. Delayed symptoms may include, but are not limited to, concentration and memory deficiencies, irritability and/or other personality changes, sensitivity to light and/or sound, changes in sleep patterns, changes to ability to taste and/or smell, and psychological adjustment issues and depression. Typically, at the time of treatment, the subject does not manifest any long term effects of a TBI such as symptoms associated with chronic traumatic encephalopathy. The subject typically does not have a tauopathy, as may be determined by medical imaging such as PET imaging for tau tangles and/or a collection of symptoms associated with a tauopathy. The subject typically also does not have a neurodegenerative disease such as but not limited to Alzheimer's disease. Those of ordinary skill in the medical arts are aware of the symptoms and physiological manifestations of neurodegenerative diseases such as Alzheimer's including brain mass abnormalities, presence and/or accumulation of beta-amyloid plaques, and the like. In some instances, the concussion itself will be diagnosed by the presence of one or more of the foregoing symptoms. The concussion may be mild, moderate or severe.

The subject that has experienced a TBI typically has experienced a traumatic event that gave rise to the TBI. Such events include but are not limited to a fall, participation in high-risk sports such as football, hockey, soccer, rugby, boxing or other contact sport, involvement in a motor vehicle collision either as a passenger or pedestrian (by-stander), involvement in a bicycle collision either as a rider or a pedestrian (by-stander), involvement in combat (e.g., as a solider or by-stander), exposure to, including close proximity to, bomb blasts, and physical abuse such as violent head shaking or blows to the head.

In some instances, the subject has experienced one or more previous TBIs.

The BBB-permeable epichaperome inhibitor may be administered orally. It may be formulated as a solid form such as a capsule, tablet, lozenge, or sublingual formulation, or as a liquid form such as a drinking solution, suspension, syrup, and the like. It may be formulated as a solid but dissolvable in liquid form, or a form that dissolves or disintegrates in the mouth or in the gastrointestinal tract following ingestion.

The BBB-permeable epichaperome inhibitor may be administered intranasally (e.g., in a nasal spray) or by inhalation (e.g., by inhaler or nebulizer).

In still other embodiments, the BBB-permeable epichaperome inhibitor may be administered intravenously or intramuscularly using an auto-injection device or system, akin to an EpiPen.

The BBB-permeable epichaperome inhibitor may be a compound having a structure of Formula I, or Formula II, or Formula III, or Formula IV, or Formula V, or Formula VIa, or Formula VIb.

The BBB-permeable epichaperome inhibitor may have a structure of Compound 1:

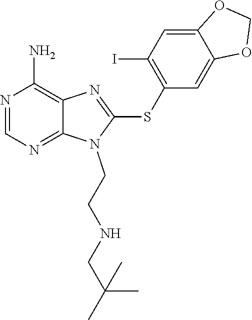

wherein I is ¹²⁷I (i.e., stable, non-decaying iodine).

In some embodiments, when used therapeutically, the BBB-permeable epichaperome inhibitor is not detectably labeled, such as for example with a radioisotope or a fluorescent moiety.

The BBB-permeable epichaperome inhibitor may be administered in an amount to reduce inflammation in the subject, including inflammation in the brain or the CNS. Such inflammation may be measured through imaging techniques such as MRI or through molecular techniques such as immune marker (e.g., an inflammatory cytokine or pro-inflammatory cytokine) or immune cell detection and measurement. It might also be administered to reduce symptoms associated with TBI as outlined above.

In some instances, the subject may be administered a second therapeutic agent such as but not limited to an anti-inflammatory agent. Administration of the BBB-permeable epichaperome inhibitor and the second therapeutic agent may be simultaneous, substantially simultaneous, or spaced in time including for example in an alternating manner. An alternating manner intends that the epichaperome inhibitor administration is followed or preceded by administration of the second therapeutic agent, and such administrations may be repeated one or more times.

The foregoing embodiments apply equally to the various aspects of this disclosure described herein, and for the sake of brevity will not be repeated.

In another aspect, provided herein is a method for reducing sequelae of traumatic brain injury (TBI) comprising administering, to a subject that has experienced a TBI, an effective amount of a BBB-permeable epichaperome inhibitor. The inhibitor may be administered within 2 weeks of the TBI in some instances. The sequelae of TBI include symptoms of TBI, including but not limited to those provided above such as headaches, nausea, dizziness, etc. The method may therefore result in reduced symptoms, reduced duration of symptoms, reduced inflammation as measured for example by the presence and amount of immune effectors and/or immune cells in the subject including in the blood, improvement in the context of a TBI or concussion scoring system such as but not limited to the Standard Assessment of Concussion, the details of which are incorporated by reference herein.

In another aspect, provided herein are a number of kits each comprising a BBB-permeable epichaperome inhibitor with instructions for use to treat a TBI as set forth herein. Certain kits comprise an oral formulation of the BBB-permeable epichaperome inhibitor. Such oral formulation may be a solid form such as a capsule, tablet, lozenge, sublingual formulation and the like, or they may be a liquid formulation such as a drinking solution, syrup, and the like. Certain kits comprise an intranasal or inhaled formulation of the BBB-permeable epichaperome inhibitor. Such intranasal or inhaled formulations may be a nasal spray, a formulation intended for administration with an inhaler or a nebulizer, and the like. Certain kits comprise a parenteral formulation of the BBB-permeable epichaperome inhibitor. Such parenteral formulations may be housed in a syringe or an auto-injection device akin to an EpiPen. The kits may include a dispensing device or system that optionally may be designed to measure the doses administered to a subject (e.g., by including a counter such as in an inhaler). Such kits may comprise additional epichaperome inhibitors, and which may or may not be BBB-permeable. Such kits may further comprise one or more secondary therapeutic agents such as but not limited to anti-inflammatory agents and an analgesic.

In some embodiments, when used therapeutically, the BBB-permeable epichaperome inhibitors are not detectably labeled, and the methods do not involve imaging of the subjects after administration of the inhibitors.

Other advantages and novel features of the present invention will become apparent from the following detailed description of various non-limiting embodiments of the invention. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### DETAILED DESCRIPTION

This disclosure is premised on the surprising finding that early intervention after a traumatic brain injury (TBI) reduces the risk of developing, delays the onset of, and/or reduces the severity of short-term and/or long-term sequelae of single or repeated TBI. More specifically, this early intervention involves the use of agents that bind selectively to Hsp90 (i.e., Hsp90 and/or Hsp90 isoforms and/or Hsp90 homologs such as but not limited to GRP94 and TRAP1) as it is complexed in an epichaperome, and are thus able to interfere with the structure and ultimately function of the epichaperome. Such epichaperome inhibitors are also selected based on their ability to cross the blood-brain-barrier (BBB), and thus they are also referred to herein as BBB-permeable epichaperome inhibitors.

These epichaperome inhibitors function, at least in part by inhibiting Hsp90 activity which in turn enhances Hsp70 activity and reduces the level of inflammation that occurs shortly after a TBI. It has not been recognized heretofore that reducing the inflammation that occurs shortly after a TBI would impact the likelihood of later developing more severe conditions such as chronic traumatic encephalopathy (CTE). There is a growing body of evidence that CTE arises many years after a subject has experienced repeated TBI. There are no current approved therapies for CTE although certain agents have been proposed to treat CTE. One advantage of the methods provided herein is the efficacy of the treatment even when administered very early after a TBI. There is no current approach to reducing the likelihood that a subject develops CTE after experiencing one or repeated TBI such as repeated concussions. This disclosure however provides such a method.

This early intervention may take place within hours of the TBI, or within days, weeks or months, and may be timed relative to the occurrence of the TBI or the occurrence of comparatively short-term (or early) symptoms associated with TBI. Subjects may be so treated after every TBI experienced by the subject. The subject may be monitored to determine the effect of the treatment on the short-term inflammation observed early after the TBI.

This disclosure provides methods for treating, including lessening the short-term and long-term effects of, traumatic brain injury (TBI) using agents that bind to Hsp90 when Hsp90 is complexed in an epichaperome, and thereby destabilize the epichaperome structure and ultimately its function. These inhibitors are able to reduce the inflammation associated with a TBI, as may be indicted by a reduction in the level of pro-inflammatory mediators and/or by an increase in the level of anti-inflammatory mediators. Suitable inhibitors are able to cross the blood-brain barrier (BBB), and are thus referred to herein as BBB-permeable. This disclosure provides methods comprising administration of certain BBB-permeable epichaperome inhibitors following a TBI. The epichaperome inhibitors may be administered early including but not limited to within an hour of the TBI. Additionally or alternatively, they may be administered repeatedly following the TBI including but not limited to one or more times a day, for 1-2 weeks or longer. In this way, the epichaperome inhibitors are able to target and thus interfere with the formation of the epichaperome. The epichaperome may begin to form early after the TBI, as a result of the stressed cellular condition. Epichaperome inhibitor administration may continue for as long as inflammation or other more overt symptoms of the TBI are present in the subject.

Certain methods and products provided herein relate to particular formulations for delivery of the BBB-permeable epichaperome inhibitors. Such formulations are those that are readily administered to a subject whether conscious or unconscious, whether a child (e.g., an infant) or an adult, whether responsive or non-responsive.

These methods provide therapeutic benefit to a subject that has experienced a TBI in one of a variety of ways including but not limited to reducing number, severity and/or duration of symptoms resulting, directly or indirectly, from such TBI, reducing inflammation in the subject and any downstream effects thereof.

### Epichaperome Inhibitors

For the sake of brevity, the term Hsp90 will be used herein to collectively refer to Hsp90, its isoforms and its homologs such as but not limited to GRP94 and TRAP1. Thus, the Hsp90 inhibitors of this disclosure inhibit Hsp90 and/or Hsp90 isoforms and/or Hsp90 homologs including but not limited to GRP94 and TRAP1. Again for the sake of brevity, inhibitors of Hsp90 (Hsp90-alpha and Hsp90-beta in the cytoplasm), Hsp90 isoforms and Hsp90 homologs, such as but not limited to GRP94 (a form of Hsp90 found in the endoplasmic reticulum) and TRAP1 (a form of Hsp90 found in the mitochondria), are referred to herein collectively as Hsp90 inhibitors or epichaperome inhibitors. More particularly, Hsp90 inhibitors that are able to cross the blood-brain barrier (BBB) are referred to as blood-brain barrier (BBB) permeable or BBB-permeable Hsp90 inhibitors.

The disclosure provides BBB-permeable epichaperome inhibitors that interfere with the formation or stability, and thus ultimately function or activity, of the epichaperome. The ability to target the epichaperome, in the context of early treatment of TBI and its downstream sequelae, may result in reduced inflammation, decreased levels of pro-inflammatory and inflammatory cytokines, increases levels of anti-inflammatory cytokines, increased levels of protective heatshock proteins, such as Hsp70, protection of neurons, decrease in formation of tau tangles and neurofibrillary tangles, decrease in beta amyloid plaque formation.

Thus, the BBB-permeable epichaperome inhibitors are defined as compounds capable of selectively binding to Hsp90 when it is complexed in an epichaperome (but binds only weakly to an uncomplexed form or in a chaperome), thereby interfering with the epichaperome stability and thus ultimately function. The ability of a compound to bind selective to Hsp90 in an epichaperome relative to Hsp90 in an uncomplexed form may be determined through standard binding assays in which the binding affinity of the compound to both forms of Hsp90 is measured. Suitable selective Hsp90 inhibitors may have at least 2-fold, 3-fold, 4-fold, 5-fold, 10-fold, 20-fold, 50-fold, 100-fold, or 1000-fold more binding affinity for epichaperome-complexed Hsp90 than uncomplexed Hsp90. For example, these inhibitors may have an EC50 in the nanomolar range for epichaperome-complexed Hsp90 and an EC50 in the micromolar range for uncomplexed or chaperome-complexed Hsp90 (as may exist in normal, unstressed cells, for example).

Certain of the inhibitors used in the methods provided herein must also be capable of crossing the blood brain barrier (BBB). Assays for determining BBB-permeability of compounds are known in the art and discussed herein.

Certain of the epichaperome inhibitors provided herein are generally referred to as purine scaffold inhibitors. One class of epichaperome inhibitors of this disclosure are purine-scaffold compounds having the general structure of Formula I:

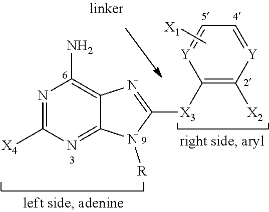

wherein each Y is independently chosen as C, N or O, with the proviso that when Y is O the double bonds are missing or rearranged to retain the aryl nature of the ring, optionally wherein both Y are C or N or O in some instances,

R is hydrogen, a C1 to C10 alkyl, alkenyl, alkynyl, or an alkoxyalkyl group, optionally including heteroatoms such as N or O, or a targeting moiety connected to N9 via a linker,

X4 is hydrogen or halogen, for example F or Cl, or Br;

X3 is CH2, CF2S, SO, SO2, O, NH, or NR2, wherein R2 is alkyl; and

X2 is halogen, alkyl, alkoxy, halogenated alkoxy, hydroxyalkyl, pyrollyl, optionally substituted aryloxy, alkylamino, dialkylamino, carbamyl, amido, alkylamido dialkylamido, acylamino, alkylsulfonylamido, trihalomethoxy, trihalocarbon, thioalkyl, SO2.alkyl, COO-alkyl, NH2, OH, CN, SO2X5, NO2, NO, C═SR2, NSO2X5, C═OR2, where X5 is F, NH2, alkyl or H, and R2 is alkyl, NH2, NH-alkyl or O-alkyl; and

X1 represents two substituents, which may be the same or different, disposed in the 4′ and 5′ positions on the aryl group, wherein X1 is selected from halogen, alkyl, alkoxy, halogenated alkoxy, hydroxyalkyl, pyrollyl, optionally substituted aryloxy, alkylamino, dialkylamino, carbamyl, amido, alkylamido dialkylamido, acylamino, alkylsulfonylamido, trihalomethoxy, trihalocarbon, thioalkyl, SO2.alkyl, COO-alkyl, NH2, OH, CN, SO2X5, NO2, NO, C═SR2NSO2X5, C═OR2, where X5 is F, NH2, alkyl or H, and R2 is alkyl, NH2, NH-alkyl or O-alkyl, C1 to C6 alkyl or alkoxy; or wherein X1 has the formula -0-(CH2)n-0-, wherein n is an integer from O to 2, and one of the oxygens is bonded at the 5′-position and the other at the 4′-position of the aryl ring.

The right-side aryl group may be phenyl as shown, or may include one or more heteroatoms. For example, the right-side aryl group may be a nitrogen-containing aromatic heterocycle such as pyrimidine.

In specific preferred embodiments of the composition of the invention, the right side aryl group X1 has the formula -0-(CH2)n-0-, wherein n is an integer from 10 to 2, preferably 1 or 2, and one of the oxygens is bonded at the 5′-position of the aryl ring and the other at the 4′ position. In other specific embodiments of the invention, the substituents X1 comprise alkoxy substituents, for example methoxy or ethoxy, at the 4′ and 5′-positions of the aryl ring.

In specific embodiments of the invention, the substituent X2 is a halogen.

In specific embodiments of the invention, the linker X3 is S. In other specific embodiments of the invention, the linker X3 is CH2.

In specific embodiments of the invention, R is a pent-4-ynyl substituent. In other specific embodiments of the invention, R contains a heteroatom, for example nitrogen. A preferred R group that increases the solubility of the compound relative to an otherwise identical compound in which R is H or pent-4-ynyl is —(CH2Xn-N—R10R11R12, where m is 2 or 3 and where R10.12 are independently selected from hydrogen, methyl, ethyl, ethene, ethyne, propyl, isopropyl, isobutyl, ethoxy, cyclopentyl, an alkyl group forming a 3 or 6-membered ring including the N, or a secondary or tertiary amine forming a 6-membered ring with the nitrogen. In specific examples, R10 and R11 are both methyl, or one of R10 and Rn is methyl and the other is ethyne.

Another class of epichaperome inhibitors of this disclosure are purine scaffold compounds having the general structure of Formula II:

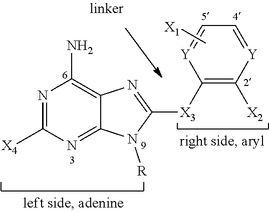

wherein R is hydrogen, a C1 to C10 alkyl, alkenyl, alkynyl, or an alkoxyalkyl group, optionally including heteroatoms such as N or O, optionally connected to the 2′-position to form an 8 to 10 member ring:

wherein the Ys are regarded as Y1 and Y2 that are independently selected as C, N, S or O, with the proviso that when Y1 and/or Y2 is O the double bonds are missing or rearranged to retain the aryl nature of the ring,

X4 is hydrogen, halogen, for example F or Cl, or Br;

X3 is CH2, CF2 S, SO, SO2, O, NH, or NR2, wherein R2 is alkyl; and

X2 is halogen, alkyl, halogenated alkyl, alkoxy, halogenated alkoxy, hydroxyalkyl, pyrollyl, optionally substituted aryloxy, alkylamino, dialkylamino, carbamyl, amido, alkylamido dialkylamido, acylamino, alkylsulfonylamido, trihalomethoxy, trihalocarbon, thioalkyl, SO2 alkyl, COO-alkyl, NH2OH, or CN or part of a ring formed by R; and

X1 represents one more substituents on the aryl group, with the proviso that X1 represents at least one substituent in the 5′-position said substituent in the 5′-position being selected from the same choices as X2 C1 to C6alkyl or alkoxy; or wherein X1 has the formula —O—(CH2)-O—, wherein n is 1 or 2, and one of the oxygens is bonded at the 5′-position of the aryl ring and the other is bonded to the 4′ position.

The ride-side aryl group may be phenyl, or may include one or more heteroatoms. For example, the right-side aryl group may be a nitrogen-containing aromatic heterocycle such as pyrimidine.

In specific embodiments of the composition of the invention, the right-side aryl group is substituted at the 2′ and 5′ position only. In other embodiment, the right side aryl group is substituted at the 2′, 4′, and 5′ positions. In yet other embodiments, the right side aryl group is substituted at the 4′ and 5′ positions only. As will be appreciated by persons skilled in the art, the numbering is based on the structure as drawn, and variations in the structure such as the insertion of a heteroatom may alter the numbering for purposes of formal nomenclature.

In other specific embodiments of the composition of the invention, the right side aryl group has a substituent at the 2′-position and X1 has the formula —X—Y—Z— with X and Z connected at the 4′ and 5′ positions to the right side aryl, wherein X, Y and Z are independently C, N, S or O, connected by single or double bonds and with appropriate hydrogen, alkyl or other substitution to satisfy valence. In some embodiments, at least one of X, Y and Z is a carbon atom. In one specific embodiment, X1 is -0-(CH2)n-O—, wherein n is 1 or 2, and one of the oxygen atoms is bonded at the 5′-position of the aryl ring and the other at the 4′ position.

In some embodiments, the compound had the structure of Formula III:

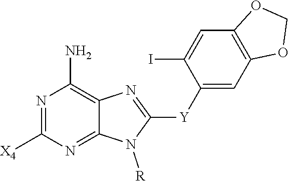

wherein:

Y is —CH2- or S,

X₄ is hydrogen or halogen and

R is an amino alkyl moiety, optionally substituted on the amino nitrogen with one or to two carbon-containing substituents selected independently from the group consisting of alkyl, alkenyl and alkynyl substituents, wherein the total number of carbons in the amino alkyl moiety is from 1 to 9, and wherein the compound is optionally in the form of an acid addition salt.

In some embodiments, R is —(CH₂)m-N—R₁₀R₁₁m, where m is 2 or 3, and R₁₀ and R₁₁ are independently selected from hydrogen, methyl, ethyl, ethenyl, ethynyl, propyl, isopropyl, t-butyl and isobutyl. In some embodiments, Y is S.

In some embodiments, R is selected from the group consisting of 2-(methyl, t-butyl amino)ethyl, 2-(methyl, isopropyl amino)ethyl, 2-(ethyl, isopropyl amino)ethyl, 3-(isopropyl amino) propyl, 3-(t-butyl amino) propyl, 2-(isopropyl amino)ethyl, 3-(ethylamino) propyl, and 3-(ethyl, methyl amino) propyl.

In some embodiments, I in the compound is ¹²⁴I, ¹³¹I or ¹²³I.

In some embodiments, I in the compound is ¹²⁷I (i.e., stable, non-decaying iodine).

Another class of epichaperome inhibitors of this disclosure have the general structure of Formula IV:

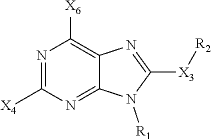

or an acid addition salt thereof,
wherein X₄ is hydrogen or halogen;

X₆ is amino;

X₃ is C, O, N, or S with hydrogens as necessary to satisfy valence, or CF₂, SO, SO₂ or NR₃ where R₃ is alkyl;

R₁ is selected from the group consisting of 3-((2-hydroxyethyl)(isopropyl)amino)propyl, 3-(methyl(prop-2-ynyl)amino)propyl, 3-(allyl(methyl)amino)propyl, 3-(cyclohexyl(2-hydroxyethylamino)propyl, 3-(4-(2-hydroxyethyl)piperazin-1-yl)propyl, 2-(isopropylamino)ethyl, 2-(isobutylamino)ethyl, or 2-(neopentylamino)ethyl, 2-(cyclopropylmethylamino)ethyl, 2-(ethyl(methyl)amino)ethyl, 2-(isobutyl(methyl)amino)ethyl, and 2-(methyl(prop-2-ynyl)amino)ethyl, or an acid addition salt thereof; and
R₂ is

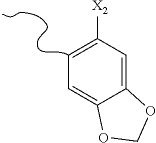

wherein X₂ is halogen.

Another class of epichaperome inhibitors of this disclosure have the general structure of Formula V:

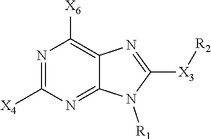

or an acid addition salt thereof,
wherein X₄ is hydrogen or halogen;

X₆ is amino;

X₃ is C, O, N, or S with hydrogens as necessary to satisfy valence, or CF₂, SO, SO₂ or NR₃ where R₃ is alkyl;

R₁ is 2-(isobutylamino)ethyl or 2-(neopentylamino)ethyl, or an acid addition salt thereof; and
R₂ is

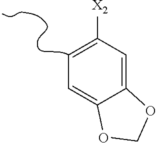

wherein X₂ is halogen.

In some embodiments, R1 is 2-(neopentylamino)ethyl.

In some embodiments, R1 is 2-(isobutylamino)ethyl.

In some embodiments, the BBB-permeable epichaperome inhibitor has the structure:

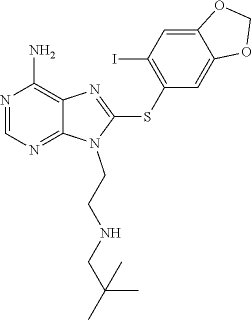

wherein I is ¹²⁷I (i.e., stable, non-decaying iodine), and is referred to herein as Compound 1.

In some embodiments, the BBB-permeable epichaperome inhibitor has the structure:

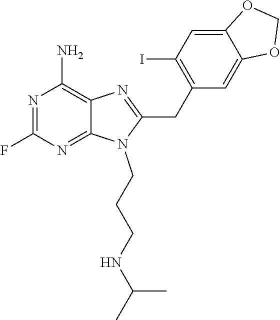

wherein F is stable, non-decaying fluorine, and I is ¹²⁷I (i.e., stable, non-decaying iodine), and is referred to as Compound 2.

In some embodiments, the BBB-permeable epichaperome inhibitor has the structure:

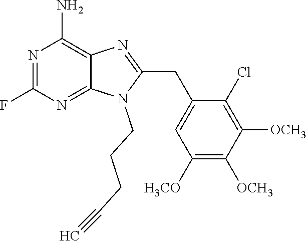

wherein F is stable, non-decaying fluorine, and is referred to as Compound 3.

Another class of epichaperome inhibitors of this disclosure have the general structure of Formula VI:

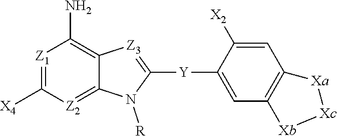

wherein
(a) each of Zl, Z2 and Z3 is independently C or N, with H substituents as needed to satisfy valence;
(b) Xa, Xb and Xc are all carbon (C), connected by two single or one single bond and one double bond,
(c) Y is —CH2- or —S—;
(d) X4 is hydrogen or halogen; and
(e) X2 and R in combination are selected from the group consisting of:

(i) X2 is halogen and R is primary amino-alkyl, a secondary or tertiary alkyl-amino-alkyl, aryl-alkyl, or a nonaromatic heterocycle-alkyl, wherein the amine's nitrogen and the heterocycle's heteroatom are substituted to satisfy valence, with the proviso that R is not a piperidine moiety; and

(ii) X2 is selected from the group consisting of alkyl, alkenyl, alkynyl, aryl, cycloalkyl, cycloalkenyl, saturated or unsaturated heterocycle, aryl, aryloxy, alkoxy, halogenated alkoxy, alkenyloxy, hydroxyalkyl, amino, alkylamine, dialkylamino, acylamino, carbamyl, amido, dialkylamido, alkylamido, alkylsulfonamido, sulfonamido, trihalocarbon, -thioalkyl, S02-alkyl, —COO-alkyl, OH or alkyl-CN, or part of a ring formed by R, and R is a group as listed below in Table A.

Another class of epichaperome inhibitors of this disclosure have the general structure of Formula VIa:

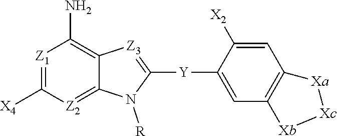

wherein

(a) each of Z1 , Z2 and Z3 is independently C or N, with H substituents as needed to satisfy valence;

(b) Xa, Xb and Xc are all carbon, connected by two single or one single bond and one double bond, and wherein

(c) Y is —CH2- or —S—;

(d) X4 is hydrogen or halogen; and

(e) X2 and R in combination are selected from the group consisting of:
(i) X2 is halogen and R is primary amino-alkyl, a secondary or tertiary alkyl-amino-alkyl, aryl-alkyl, or a nonaromatic heterocycle-alkyl, wherein the amine' s nitrogen and the heterocycle's heteroatom are substituted to satisfy valence, with the proviso that R is not a piperidino moiety; and
(ii) X2 is selected from the group consisting of alkyl, alkenyl, alkynyl, aryl, cycloalkyl, cycloalkenyl, saturated or unsaturated heterocycle, aryl, aryloxy, alkoxy, halogenated alkoxy, alkenyloxy, hydroxyalkyl, amino, alkylamino, dialkylamino, acylamino, carbamyl, amido, dialkylamido, alkylamido, alkylsulfonamido, sulfonamido, trihalocarbon, -thioalkyl, S02-alkyl, —COO-alkyl, OH or alkyl-CN, or part of a ring formed by R, and R is a group listed in Table A.

In some embodiments of Formula VIa, X2 is not halogen.

In some embodiments of Formula VIa, X2 is alkynyl.

In some embodiments of Formula VIa, the compound is selected from the group consisting of: 8-((6-ethynyl-2,3-dihydro-1H-inden-5-yl)thio)-9-(3-(isopropylamino)propyl)-9H-purin-6-amine; 1-(3-(2-(6-amino-8-(6-ethynyl-2,3-dihydro-1H-inden-5-ylthio)-9H-purin-9-yl)ethyl)piperidin-1-yl)ethanone; 1-(3-(3-(6-amino-8-(6-ethynyl-2,3-dihydro-1H-inden-5-ylthio)-9H-purin-9-yl)propyl)pyrrolidin-1-yl)ethanone; 8-((6-ethynyl-2,3-dihydro-1H-inden-5-yl)thio)-9-(2-(neopentylamino)ethyl)-9H-purin-6-amine; 5-(6-amino-8-(6-ethynyl-2,3-dihydro-1H-inden-5-ylthio)-9H-purin-9-yl)pentane-1-sulfonamide; 1-(4-(3-(6-amino-8-(6-ethynyl-2,3-dihydro-1H-inden-5-ylthio)-9H-purm-9-yl)propyl)piperidin-1-yl)ethanone; 9-(3-(tert-butylamino)propyl)-8-(6-ethynyl-2,3-dihydro-1H-inden-5-ylthio)-9H-purin-6-amine; 1-acetyl-3-(3-(6-amino-8-(6-ethynyl-2,3-dihydro-1H-inden-5-ylthib)-9H-purin-9-yl)propyl)imidazolidin-2-one; 8-((6-ethynyl-2,3-dihydro-1H-inden-5-yl)thio)-9-(2-(1-methylpiperidin-2-yl)ethyl)-9H-purin-6-amine; 8-((6-ethynyl-2,3-dihydro-1H-inden-5-yl)thio)-9-(2-(1-methylpiperidin-3-yl)ethyl)-9H-purin-6-amine; 8-((6-ethynyl-2,3-dihydro-1H-inden-5-yl)thio)-9-(2-(1-(methylsulfonyl)piperidin-3-yl)ethyl)-9H-purin-6-amine; 1-(3-(2 6-amino-8-((6-ethynyl-2,3-dihyo\hat{ }o H-inden-5-yI)methyl)-2-fluoro-9H-purin-9-yl)ethyl)piperidin-1-yl)ethanone; 9-(3-(tert-butylamino)propyl)-8-((6-ethynyl-2,3-dihydro-1H-inden-5-yl)methyl)-2-fluoro-9H-purin-6-amine; 6-(6-amino-8-((6-ethynyl-2,3-dihydro-1H-inden-5-yl)methyl)-2-fluoro-9H-purin-9-yl)hexanamide; 1-(3-(6-amino-8-((6-ethynyl-2,3-dihydro-1H-inden-5-yl)methyl)-2-fluoro-9H-purin-9-yl)propyl)pyrrolidin-3-one; 4-(6-amino-8-((6-ethynyl-2)3-dihydro-1H-inden-5-yl)methyl)-2-fluoro-9H-purin-9-yl)butane-1-sulfonamide; 8-((6-ethynyl-2,3-dihydro-1H-inden-5-yl)methyl)-2-fiuoro-9-(3-(isopropylamino)propyl)-9H-purin-6-amine; 8-((6-ethynyl-2,3-dihydro-1H-inden-5-yl)methyl)-2-fluoro-9-(2-(neopentylamino)ethyl)-9H-purin-6-amine; 3-(2-(6-amino-8-((6-ethynyl-2,3-dihydro-1H-inden-5-yl)methyl)-2-fluoro-9H-purin-9-yl)ethyl)piperidine-1-sulfonamide; 8-((6-ethynyl-2,3-dihydro-1H-inden-5-yl)methyl)-2-fluoro-9-(2-(1-methylpiperidin-2-yl)ethyl)-9H-purin-6-amine; and 8-((6-ethynyl-2,3-dihydro-1H-inden-5-yl)methyl)-2-fluoro-9-(2-(1-methylpiperidin-3-yl)ethyl)-9H-purin-6-amine.

In some embodiments of Formula VIa, X2 is heteroaryl.

In some embodiments of Formula VIa, the compound is selected from the group consisting of: 8-((6-(furan-2-yl)-2,3-dihydro-1H-inden-5-yl)thio)-9-(3-(isopropylamino)propyl)-9H-purin-6-amine; 9-(3-(isopropylamino)propyl)-8-((6-(oxazol-2-yl)-2,3-dihydro-1H-inden-5-yl)thio)-9H-purin-6-amine; 1-(3-(2-(6-amino-8-(6-(oxazol-2-yl)-2,3-dihydro-1H-inden-5-ylthio)-9H-purin-9-yl)ethyl)piperidin-1-yl)ethanone; 3-(2-(8-(6-(1H-pyrazol-3-yl)-2,3-dihydro-1H-inden-5-ylthio)-6-arrimo-9H-purin-9-yl)ethyl)pipericarbaldehyde; N-(2-((2-(6-amino-8-((6-(oxazol-2-yl)-2,3-dihydro-1H-inden-5-yl)thio)-9H-purin-9-yl)ethyl)amino)ethyl)sulfamide; 3-(2-(6-amino-8-(6-(oxazol-2-yl)-2,3-dihydro-1H-inden-5-ylthio)-9H-purin-9-yl)ethylamino)-N-hydroxypropanamide; 9-(3-(isopropylamino)propyl)-8-((6-(5-methyloxazol-2-yl)-2,3-dihydro-1H-inden-5-yl)thio)-9H-purin-6-amine; 8-((6-(5-methyloxazol-2-yl)-2,3-dihydro-1H-inden-5-yl)thio)-9-(2-(1-(methylsulfonyl)piperidin-3-yl)ethyl)-9H-purin-6-amine; 9-(3-aminopropyl)-8-((6-(5-methyloxazol-2-yl)-2,3-dihydro-1H-inden-5-yl)thio)-9H-purin-6-amine; 9-(3-(tert-bu1ylamino)propyl)-8-(6-(4-memyltm\hat{ }ol-2-yl)-2,3-dihydro-1H-inden-5-ylthio)-9H-purin-6-amine; 8-((6-(5-methyloxazol-2-yl)-2,3-dihydro-1H-inden-5-yl)thio)-9-(2-(neopentylaniino)ethyl)-9H-purin-6-amine; 1-(6-amino-8-((6-(5-methyloxazol-2-yl)-2,3-dihydro-1H-inden-5-yl)thio)-9H-purin-9-yl)-3-(isopropylamino)propan-2-ol; 1-(2-(4-(6-amino-8-(6-(5-methylfuran-2-yl)-2,3-dihydro-1H-inden-5-ylthio)-9H-purin-9-yl)butyl)pyrrolidin-1-yl)ethanone; 1-(3-(2-(6-amino-8-(6-(5-methyloxazol-2-yl)-2,3-dihydro-1H-inden-5-ylthio)-9H-purin-9-yl)ethyl)piperidin-1-yl)ethanone; 6-(6-amino-8-(6-(oxazol-2-yl)-2,3-dihydro-1H-inden-5-ylthio)-9H-purin-9-yl)hexanamide; 1-(3-(6-amino-8-(6-(4-methyloxa2; ol-2-yl)-2,3-dihydro-1H-inden-5-ylthio)-9H-purin-9-yl)propyl)pyrrolidin-3-one; 2-fiuoro-9-(3-(1-(methylsulfonyl)pyrrolidin-3-yl)propyl)-8-((6-(oxazol-2-yl)-2,3-dihydro-1H-inden-5-yl)methyl)-9H-purin-6-amine; 1-(3-(2-(6-amino-2-fluoro-8-((6-(4-methylthiazol-2-yl)-2,3-dihydro-1H-inden-5-yl)methyl)-9H-purin-9-yl)ethyl)piperidin-1-yl)ethanone; 9-(3-(tert-butylamino)propyl)-2-fluoro-8-((6-(4-memylthiazol-2-yl)-2,3-dihydro-1H-inden-5-yl)methyl)-9H-purin-6-amine; 8-((6-(1H-pyrazol-3-yl)-2,3-dihydro-1H-inden-5-yl)methyl)-9-(3-(tert-butylarmno)propyl)-2-fluoro-9H-purin-6-arnine; 6-(6-amino-2-fluoro-8-((6-(oxazol-2-yl)-2,3-dihydro-1H-inden-5-yl)methyl)-9H-purin-9-yl)hexanamide; 1-(3-(6-amino-2-fluoro-8-((6-(oxazol-2-yl)-2,3-dihydro-1H-inden-5-yl)methyl)-9H-purin-9-yl)propyl)pyrrolidin-3-one; 5-(6-amino-2-fluoro-8-((6-(oxazol-2-yl)-2,3-dihydro-1H-inden-5-yl)methyl)-9H-purin-9-yl)pentane-1-sulfonamide; 2-fluoro-9-(2-(1-methylpiperidin-2-yl)ethyl)-8-((6-(oxazol-2-yl)-2,3-dihydro-1H-inden-5-yl)methyl)-9H-purin-6-amine; and 2-fluoro-9-(2-(1-methylpiperidin-3-yl)ethyl)-8-((6-(oxazol-2-yl)-2,3-dihydro-1H-inden-5-yl)methyl)-9H-purin-6-amine.

In some embodiments of Formula VIa, X2 is iodine.

In some embodiments, the Hsp90 inhibitor is selected from the group consisting of: 1-(6-amino-8-(6-iodo-2,3-dihydro-1H-inden-5-ylthio)-9H-purin-9-yl)-3-(tert-butylamino)propan-2-ol; 8-((6-iodo-2,3-dihydro-1H-inden-5-yl)thio)-9-(2-(isobutylamino)ethyl)-9H-purin-6-amine; 1-(3-(6-amino-8-(6-iodo-2,3-dihydro-1H-inden-5-ylthio)-9H-purm-9-yl)propyl)pyrrolidin-3-one; 1-(3-(3-(6-amino-8-(6-iodo-2,3-dihydro-1H-inden-5-ylthio)-9H-purin-9-yl)propyl)pyrrolidin-1-yl)ethanone; 8-((6-iodo-2,3-dihydro-1H-inden-5-yl)thio)-9-(2-(neopentylamino)ethyl)-9H-purin-6-amine; 8-((6-iodo-2,3-dihydro-1H-inden-5-yl)thio)-9-(3-(isopropylamino)propyl)-9H-purin-6-amine; 9-(3-aminopropyl)-8-((6-iodo-2,3-dihydro-1H-inden-5-yl)thio)-9H-purin-6-amine; 9-(2-aminoethyl)-8-((6-iodo-2,3-dihydro-1H-inden-5-yl)thio)-9H-purin-6-amine; 9-(3-(tert-butylamino)propyl)-8-((6-iodo-2,3-dihydro-1H-inden-5-yl)thio)-9H-purin-6-amine; 5-(6-amino-8-(6-iodo-2,3-dihydro-1H-inden-5-ylthio)-9H-purin-9-yl)-N-methylpentane-1-sulfonamide; 5-(6-amino-8-(6-iodo-2,3-dihydro-1H-inden-5-ylthio)-9H-purin-9-yl)pentane-1-sulfonamide; 1-(3-(6-amino-8-(6-iodo-2,3-dihydro-1H-inden-5-ylthto)-9H-purin-9-yl)propyl)pyrrolidin-3-ol; 6-(6-amino-8-(6-iodo-2,3-dihydro-1H-inden-5-ylthio)-9H-purin-9-yl)hexanamide; 8-((6-iodo-2,3-dihydro-1H-inden-5-yl)thio)-9-(2-(1-methylpiperidin-2-yl)ethyl)-9H-purin-6-amine; 8-((6-iodo-2,3-dihydro-1H-inden-5-yl)thio)-9-(2-(1-methylpiperidin-3-yl)ethyl)-9H-purin-6-amine; 8-((6-iodo-2,3-dihydro-1H-inden-5-yl)thio)-9-(2-(1-(methylsulfonyl)piperidin-3-yl)ethyl)-9H-purin-6-amine; 3-(2-(6-amino-8-((6-iodo-2,3-dihydro-1H-inden-5-yl)thio)-9H-purin-9-yl)ethyl)piperidine-1-sulfonamide; 2-fiuoro-8-((6-iodo-2,3-dihydro-1H-inden-5-yl)methyl)-9-(2-(isobutylamino)ethyl)-9H-purin-6-amine; 2-fluoro-8-((6-iodo-2,3-dihydro-1H-inden-5-yl)methyl)-9-(3-(isopropylamino)propyl)-9H-purin-6-amine; 1-(3-(6-amino-2-fluoro-8-((6-iodo-2,3-dmydro-m-inden-5-yl)methyl)-9H-purin-9-yl)pr0pyl)pyiToli 1-(3-(3-(6-amino-2-fluoro-8-((6-iodo-2,3-dihydro-1H-inden-5-yl)methyl)-9H-purin-9-yl)propyl)pyrrolidin-1-yl)ethanone; 9-(3-(tert-butylamino)propyl)-2-fluoro-8-((6-iodo-2,3-dihydfo-1H-inden-5-yl)methyl)-9H-purin-6-amine; 5-(6-amino-2-fiuoro-8-((6-iodo-2,3-dihydro-1H-inden-5-yl)methyl)-9H-purin-9-yl)-N-methylpentane-1-sulfonamide; 5-(6-amino-2-fluoro-8-((6-iodo-2,3-dihydro-1H-inden-5-yl)methyl)-9H-purin-9-yl)pentane-1-sulfonamide; 2-fluoro-8-((6-iodo-2,3-dihydro-1H-inden-5-yl)methyl)-9-(2-(1-methylpiperidin-2-yl)ethyl)-9H-purin-6-amine; 2-fluoro-8-((6-iodo-2,3-dihydro-1H-inden-5-yl)methyl)-9-(2-(1-methylpiperidin-3-yl)ethyl)-9H-purin-6-amine; 2-fluoro-8-((6-iodo-2,3-dihydro H-inden-5-yl)methyl)-9-(2-(1-(methylsulfonyI)piperidin-3-yl)ethyl)-9H-purin-6-amine; 3-(2-(6-amino-2-fluoro-8-((6-iodo-2,3-dihydro-1H-inden-5-yl)methyl)-9H-purin-9-yl)ethyl)piperidine-1-sulfonamide; and 9-(3-(tert-butylamino)propyl)-2-fluoro-8-((6-iodo-2,3-dihydro-1H-inden-5-yl)methyl)-9H-purin-6-amine.

Another class of epichaperome inhibitors of this disclosure have the general structure of Formula VII:

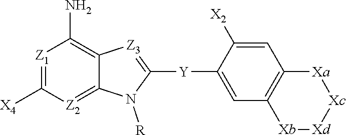

wherein

(a) each of Z1, Z2 and Z3 is independently C or N, with H substituents as needed to satisfy valence;

(b) Xa and Xb are O, and Xc and Xd are CH2;

(c) Y is —CH2-, —O— or —S—;

(d) X4 is hydrogen or halogen; and

(e) X2 and R are a combination selected from:
(i) X2 is halogen or cyano and R is suitably a primary amino alkyl, a secondary or tertiary alkyl-amino-alkyl, a trialkylammonioalkyl group, an aryl-alkyl, or a nonaromatic heterocycle-alkyl, with the proviso that R does not include a piperidino moiety; and
(ii) X2 is selected from the group consisting of an aryl, an alkynyl, a cycloalkyl and an cycloalkenyl; and R is a group listed in Table A.

In some embodiments of Formula VII, X2 is halogen.

In some embodiments of Formula VII, X2 is iodine.

In some embodiments, the Hsp90 inhibitor is selected from the group consisting of: 8-((7-iodo-2,3-dihydrobenzo[b][1,4]dioxin-6-yl)thio)-9-(3-(isopropylamino)propyl)-9H-purin-6-amine; 8-((7-iodo-2,3-dihydrobenzo[b][1,4]dioxin-6-yl)thio)-9-(2-(isobutylamino)ethyl)-9H-purin-6-amine; 8-((7-iodo-2,3-dihydrobenzo[b][1,4]dioxin-6-yl)thio)-9-(2-(neopentylann\hat{ }o)emyl)-9H-purm-6-amine; 9-(3-(1H-imidazol-1-yl)propyl)-8-((7-iodo-2,3-dihydrobenzo[b][1,4]dioxin-6-yl)thio)-9H-purin-6-amine; 9-(3-aminopropyl)-8-((7-iodo-2,3-dihydrobenzo[b][1,4]dioxin-6-yl)thio)-9H-purin-6-amine; 9-(2-aminoethyl)-8-((7-iodo-2,3-dihydrobenzo[b][1,4]dioxin-6-yl)thio)-9H-purin-6-amine; 9-(3-(tert-butylarmno)propyl)-8-((7-iodo-2,3-dihydrobenzo[b][1,4]dioxin-6-yl)thio)-9H-purin-6-amine; 1-(6-amino-8-((7-iodo-2,3-dihydrobenzo[b][1,4]dioxin-6-yl)thio)-9H-purin-9-yl)-3-(isopropylamino)propan-2-ol; 5-(6-amino-8-(7-iodo-2,3-dihydrobenzo[b][1,4]dioxin-6-ylthio)-9H-purin-9-yl)pentane-1-sulfonamide; 1-(3-(6-amino-8-(7-iodo-2,3-dihydrobenzo[b][1,4]dioxin-6-ylthio)-9H-purin-9-yl)propyl)pyiTolidin-3-one; 6-(6-amino-8-(7-iodo-2,3-dihydrobenzo[b][1,4]dioxin-6-ylthio)-9H-purin-9-yl)hexanamide; 1-(3-(4-(6-amino-8-(7-iodo-2,3-dihydrobenzo[b][1,4]dioxin-6-ylthio)-9H-purin-9-yl)butyl)pyrrolidin-1-yl)ethanone; and 8-(7-iodo-2,3-dihydrobenzo[b][1,4]dioxin-6-ylthio)-9-(3-(isobutylamino)propyl)-9H-purin-6-amine.

In some embodiments of Formula VII, X2 is heteroaryl. In some embodiments of Formula VII, X2 is pyrazole.

In some embodiments, the epichaperome inhibitor is selected from the group consisting of: 8-((7-(1H-pyrazol-3-yl)-2,3-dihydrobenzo[b][1,4]dioxin-6-yl)thio)-9-(3-(isopropylamino)propyl)-9H-purin-6-amine; 8-((7-(1H-pyrazol-3-yl)-2,3-dihydrobenzo[b][1,4]dioxin-6-yl)thio)-9-(2-(neopentylamino)ethyl)-9H-purin-6-amine; 1-(4-(2-(8-((7-(1H-pyrazol-3-yl)-2,3-dihydrobenzo[b][1,4]dioxin-6-yl)thio)-6-amino-9H-purin-9-yl)ethyl)piperidin-1-yl)ethanone; 8-(7-(1H-pyrazol-3-yl)-2,3-dihydrobenzo[b][1,4]dioxin-6-ylthio)-9-(2-(1-(methylsulfonyl)piperidin-3-yl)ethyl)-9H-purin-6-amine; N-(2-((2-(8-((7-(1H-pyrazol-3-yl)-2,3-dihydrobenzo[b][1,4]dioxin-6-yl)thio)-6-amino-9H-purin-9-yl)ethyl)amino)ethyl)sulfamide; 8-((7-(1H-pyrazol-3-yl)-2,3-dihydrobenzo[b][1,4]dioxin-6-yl)thio)-9-(3-aminopropyl)-9H-purin-6-amine; 8-((7-(1H-pyrazol-3ryl)-2,3-dihydrobenzo[b][1,4]dioxin-6-yl)thio)-9-(3-(tert-butylamino)propyl)-9H-purm-6-amm\hat{ } 9-(3-(isopropylamino)propyl)-8-((7-(5-methyl-1H-pyrazol-3-yl)-2,3-dihydrobenzo[b][1,4]dioxin-6-yl)thio)-9H-purin-6-amine; 8-((7-(5-methyl-1H-pyrazol-3-yl)-2,3-dihydrobenzo[b][1,4]dioxin-6-yl)thio)-9-(2-(neopentylamino)ethyl)-9H-purin-6-amine; 1-(8-((7-(1H-pyrazol-3-yl)-2,3-dihydrobenzo[b][1,4]dioxin-6-yl)thio)-6-amino-9H-purin-9-yl)-3-(isopropylamino)propan-2-ol; 5-(8-(7-(1H-pyrazol-3-yl)-2)3-dihydrobenzo[b][1,4]dioxin-6-ylthio)-6-amino-9H-purin-9-yl)pentane-1-sulfonamide; 6-(8-(7-(1H-pyrazol-3-yl)-2,3-dihydrobenzo[b][1,4]dioxin-6-ylthio)-6-amino-9H-purin-9-yl)hexanamide; 1-(3-(8-(7-(1H-pyrazol-3-yl)-2,3-dihydrobenzo[b3[1,4]dioxin-6-ylthio)-6-amino-9H-purin-9-yl)propyl)pyrrolidin-3-one; 8-((7-(1H-pyrazol-3-yl)-2,3-dihydrobenzo[b][1,4]dioxin-6-yl)methyl)-2-fluoro-9-(2-(isobutylarmno)ethyl)-9H-purin-6-amine; 1-(4-(2-(8-((7-(1H-pyrazol-3-yl)-2,3-dihydrobenzo[b][1,4]dioxin-6-yl)methyl)-6-amino-2-fluoro-9H-purin-9-yl)ethyl)piperidin-1-yl)ethanone; 1-(3-(2-(8-((7-(1H-pyrazol-3-yl)-2,3-dihydrobenzo[b][1,4]dioxin-6-yl)methyl)-6-amino-2-fluoro-9H-purin-9-yl)emyl)piperidin-1-yl)ethanone; 8-((7-(1H-pyrazol-3-yl)-2,3-dihydrobenzo[b][1,4]dioxin-6-yl)methyl)-2-fluoro-9-(2-(1-(methylsulfonyl)piperidin-3-yl)ethyl)-9H-purin-6-amine; 1-(3-(8-((7-(1H-pyrazol-3-yl)-2,3-dihydrobenzo[b][1,4]dioxin-6-yl)methyl)-6-amino-2-fluoro-9H-purin-9-yl)propyl)pyrrolidin-3-one; 8-((7-(1H-pyrazol-3-yl)-2,3-dihydrobenzo[b][1,4]dioxin-6-yl)methyl)-9-(3-(tert-butylamino)propyl)-2-fluoro-9H-purin-6-amine; 1-(8-((7-(1H-pyrazol-3-yl)-2,3-dihydrobenzo[b][1,4]dioxin-6-yl)methyl)-6-amino-2-fluoro-9H-purin-9-yl)-3-(tert-butylamino)propan-2-ol; 5-(8-((7-(1H-pyrazol-3-yl)-2,3-dihydrobenzo[b][1,4]dioxin-6-yl)methyl)-6-amino-2-fluoro-9H-purin-9-yl)pentane-1-sulfonamide; 6-(8-((7-(1H-pyrazol-3-yl)-2,3-dihydrobenzo[b][1,4]dioxin-6-yl)methyl)-6-amino-2-fluoro-9H-purin-9-yl)hexanamide; and 8-((7-(1H-pyrazol-3-yl)-2,3-dihydrobenzo[b][1,4]dioxin-6-yl)methyl)-9-(2-aminoethyl)-2-fluoro-9H-purin-6-amine.

In some embodiments of Formula VII, X2 is a furan.

In some embodiments, the epichaperome inhibitor is selected from the group consisting of: 8-((7-(furan-2-yl)-2,3-dihydrobenzo[b][1,4]dioxin-6-yl)thio)-9-(3-(isopropylamino)propyl)-9H-purin-6-amine; 9-(3-(isopropylamino)propyl)-8-((7-(5-methylflu-an-2-yl)-2,3-cUhydrobenzo[b][1,4]dioxin-6-yl)thio)-9H-purin-6-amine; 8-((7-(5-methylfuran-2-yl)-2,3-dihydrobenzo[b][1,4]dioxin-6-yl)thio)-9-(2-(neopentylamino)ethyl)-9H-purin-6-amine; 8-((7-(5-(ammomethyl)furan-2-yl)-2,3-dihydrobenzo[b][1,4]dioxin-6-yl)thio)-9-(2-(neopentylamino)ethyl)-9H-purin-6-amine; 8-(7-(5-methylfuran-2-yl)-2,3-dihydrobenzo[b][1,4]dioxin-6-ylthio)-9-(2-(1-(methylsulfonyl)piperidin-3-yl)ethyl)-9H-purin-6-amine; 1-(3-(2-(6-ammo-8-(7-(5-memylfuran-2-yl)-2,3-dihydrobenzo[b][1,4]dioxin-6-ylthio)-9H-purin-9-yl)ethyl)piperidin-1-yl)ethanone; 1-(4-(2-(6-amino-8-((7-(5-methylfuran-2-yl)-2,3-dihydrobenzo[b][1,4]dioxin-6-yl)thio)-9H-purin-9-yl)ethyl)piperidin-1-yl)ethanone; 1-(3-(2-(6-amino-8-(7-(5-(aminomethyl)furan-2-yl)-2,3-dihydrobenzo[b][1,4]dioxin-6-ylthio)-9H-purin-9-yl)ethyl)piperidin-1-yl)ethanone; 5-(6-amino-8-(7-(5-methylraran-2-yl)-2,3-dihydrobenzo[b][1,4]dioxin-6-ylthio)-9H-purin-9-yl)pentane-1-sulfonamide; 1-(3-(6-amino-8-(7-(5-methylfuran-2-yl)-2,3-dihydrobenzo[b][1,4]dioxin-6-ylthio)-9H-purin-9-yl)propyl)pyrrolidin-3-one; 1-(6-amino-8-((7-(5-methylfuran-2-yl)-2,3-dihydrobenzo[b][1,4]dioxin-6-yl)thio)-9H-purin-9-yl)-3-(isopropylamino)propan-2-ol; 9-(3-aminopropyl)-8-(7-(5-methylfuran-2-yl)-2,3-dihydrobenzo[b][1,4]dioxin-6-ylthio)-9H-purin-6-amine; N-(2-((2-(6-amino-8-((7-(furan-2-yl)-2,3-dihydrobenzo[b][1,4]dioxin-6-yl)tWo)-9H-purin-9-yl)ethyl)amino)emyl)sul& 3-((2-(6-amino-8-((7-(furan-2-yl)-2,3-dihydrobenzo[b][1,4]dioxin-6-yl)thio)-9H-purin-9-yl)ethyl)amino)-N-hydroxypropanamide; 9-(3-(tert-butylamino)propyl)-8-(7-(5-methylfuran-2-yl)-2,3-dihydrobenzo[b][1,4]dioxin-6-ylthio)-9H-purin-6-amine; 6-(6-amino-2-fluoro-8-((7-(5-methyloxazol-2-yl)-2,3-Hhydrobenzo[b][1,4]dioxin-6-yl)methyl)-9H-purin-9-yl)hexanamide; 2-fluoro-8-((7-(5-methylfuran-2-yl)-2,3-dihydrobenzo[b][1,4]dioxin-6-yl)methyl)-9-(2-(1-(methylsulfonyl)piperidin-3-yl)ethyl)-9H-purin-6-amine; 1-(3-(2-(6-amino-2-fluoro-8-((7-(5-methylfuran-2-yl)-2,3-dihydrobenzo[b][1,4]dioxin-6-yl)methyl)-9H-purin-9-yl)ethyl)piperidin-1-yl)ethanone; 1-(4-(2-(6-amino-2-fiuoro-8-((7-(5-methylfuran-2-yl)-2,3-dihydrobenzo[b][1,4]dioxin-6-yl)methyl)-9H-purin-9-yl)ethyl)piperidin-1-yl)ethanone; 1-(3-(2-(6-amino-8-((7-(5-(aminomethyl)furan-2-yl)-2,3-dihydrobenzo[b][1,4]dioxin-6-yl)methyl)-2-fluoro-9H-purin-9-yl)ethyl)piperidin-1-yl)ethanone; 2-fluoro-8-((7-(furan-2-yl)-2,3-dihydrobenzo[b][1,4]dioxin-6-yl)methyl)-9-(2-(isobutylamino)ethyl)-9H-purin-6-amine; 2-fluoro-9-(2-(isobutylamino)ethyl)-8-((7-(5-methylfuran-2-yl)-2,3-dihydrobenzo[b][1,4]dioxin-6-yl)methyl)-9H-purin-6-amine 8-((7-(5-(aminomethyl)ftiran-2-yl)-2,3-dihydrobenzo[b][1,4]dioxin-6-yl)methyl)-2-fluoro-9-(2-(isobutylamino)ethyl)-9H-purin-6-amine; 1-(3-(6-amino-2-fluoro-8-((7-(5-methyloxazol-2-yl)-2,3-dihydrobenzo[b][1,4]dioxin-6-yl)methyl)-9H-purin-9-yl)propyl)pyrrolidin-3-one; 2-chloro-8-((7-(5-methylfuran-2-yl)-2,3-dihydrobenzo[b][1,4]dioxin-6-yl)methyl)-9(methylsulfonyl)pyrrolidin-3-yl)ethyl)-9H-purin-6-amine; 9-(3-aminopropyl)-2-fluoro-8-((7-(5-methylfuran-2-yl)-2,3-dihydrobenzo[b][1,4]dioxin-6-yl)methyl)-9H-purin-6-amine; 5-(6-ammo-2-fluoro-8-((7-(5-methylfuran-2-yl)-2,3-dihydrobenzo[b][1,4]dioxin-6-yl)methyl)-9H purin-9-yl)pentane-1-sulfonamide; and 6-(6-amino-2-fluoro-8-((7-(5-methylfuran-2-yl)-2,3-dihydrobenzo[b][1,4]dioxin-6-yl)methyl)-9H-puiin-9-yl)hexanamide.

In some embodiments of Formula VII, X2 is an oxazole.

In some embodiments, the epichaperome inhibitor is selected from the group consisting of: 1-(3-(6-amino-8-(7-(oxazol-2-yl)-2,3-dihydrobenzo[b][1,4]dioxin-6-ylthio)-9H-purin-9-yl)propyl)pyrrolidin-3-one; 6-(6-amino-8-(7-(5-methyloxazol-2-yl)-2,3-dihydrobenzo[b][1,4]dioxin-6-ylthio)-9H-purin-9-yl)hexanamide; 8-(7-(5-methyloxazol-2-yl)-2,3-dmydrobenzo[b][1,4]dioxin-6-ylthio)-9-(2-(neopentylamino)ethyl)-9H-purin-6-amine; 1-(3-(2-(6-amino-8-(7-(5-methyloxazol-2-yl)-2,3-dihydrobenzo[b][1,4]dioxin-6-ylthio)-9H-purin-9-yl)ethyl)piperidin-1-yl)ethanone; 1-(4-(2-(6-amino-8-((7-(5-methyloxazol-2-yl)-2,3-dihydrobenzo[b][1,4]dioxin-6-yl)thio)-9H-purin-9-yl)ethyl)piperi 1-yl)ethanone; 8-((7-(5-methyloxazol-2-yl)-2,3-dihydrobenzo[b][1,4]dioxin-6-yl)thio)-9-(2-(1-(methylsulfonyl)piperidin-3-yl)ethyl)-9H-purin-6-amine; 5-(6-amino-8-(7-(5-methyloxazol-2-yl)-2,3-dihydrobenzo[b][1,4]dioxin-6-ylthio)-9H-purin-9-yl)pentane-1-sulfonamide; N-(3-(6-amino-8-((7-(5-methyloxazol-2-yl)-2,3-dihydrobenzo[b][1,4]dioxin-6-yl)thio)-9H-purin-9-yl)propyl)methanesulfonamide; 1-(2-(4-(6-amino-8-(7-(5-methyloxazol-2-yl)-2,3-dihydrobenzo[b][1,4]dioxin-6-ylthio)-9H-purin-9-yl)butyl)pyrrolidin-1-yl)ethanone; 1-(6-amino-8-((7-(5-methyloxazol-2-yl)-2,3-dihydrobenzo[b][1,4]dioxin-6-yl)thio)-9H-purin-9-yl)-3-(isopropylamino)propan-2-ol; 9-(3-(tert-butylamino)propyl)-8-((7-(oxazol-2-yl)-2,3-dihydrobenzo[b][1,4]dioxin-6-yl)thio)-9H-purin-6-amine; 9-(3-aminopropyl)-8-((7-(oxazol-2-yl)-2,3-dihydrobenzo|¾][1,4]dioxin-6-yl)thio)-9H-purin-6-amine; 8-((7-(furan-2-yl)-2,3-dihydrobenzo[b][1,4]dioxin-6-yl)thio)-9-(2-(isobutylamino)ethyl)-9H-purin-6-amine; 9-(3-(isopropylamino)propyl)-8-((7-(oxazol-2-yl)-2,3-dihydrobenzo[b][1,4]dioxin-6-yl)thio)-9H-purin-6-amine; 1-(2-(4-(6-amino-8-(7-(5-methyloxazol-2-yl)-2,3-dihy<kobenzo[b][1,4]dioxin-6-yltWo)-9H-purin-9-yl)butyl)pyrrolidm 1-yl)ethanone; 1-(4-(2-(6-amino-8-((7-(5-methyloxazol-2-yl)-2,3-dihydrobenzo[b][1,4]dioxin-6-yl)thio)-9H-purin-9-yl)ethyl)piperidin-1-yl)ethanone; 8-((7-(5-methyloxazol-2-yl)-2,3-dihydrobenzo[b][1,4]dioxin-6-yl)thio)-9-(2-(1-(methylsulfonyl)piperidin-3-yl)ethyl)-9H-purin-6-amine; 2-fluoro-9-(3-(isopropylamino)propyl)-8-((7-(oxazol-2-yl)-2,3-dihydrobenzo[b][1,4]dioxin-6-yl)methyl)-9H-purin-6-amine; 2-fluoro-9-(3-(isopropylamino)propyl)-8-((7-(5-methyloxazol-2-yl)-2,3-dihydrobenzo[b][1,4]dioxin-6-yl)methyl)-9H-purin-6-amine; 9-(3-(tert-butylamino)propyl)-2-fluoro-8-((7-(oxazol-2-yl)-2,3-dihydrobenzo[b][1,4]dioxin-6-yl)methyl)-9H-purin-6-amine; 9-(3-(tert-butylamino)propyl)-2-fluoro-8-((7-(5-methyloxazol-2-yl)-2,3-dihydrobenzo[b][1,4]dioxin-6-yl)meth.yl)-9H-purin-6-amine; 6-(6-amino-2-fluoro-8-((7-(5-methyloxazol-2-yl)-2,3-dihydrobenzo[b][1,4]dioxin-6-yl)methyl)-9H-purin-9-yl)hexanamid\hat{ } 5-(6-amino-2-fluoro-8-((7-(5-methyloxazol-2-yl)-2,3-dihydrobenzo[b][1,4]dioxin-6-yl)methyl)-9H-purin-9-yl)pentane-1-sulfonamide; 1-(3-(6-amino-2-fluoro-8-((7-(5-methyloxazol-2-yl)-2,3-dihydrobenzo[b][1,4]dioxin-6-yl)methyl)-9H-piirin-9-yl)propyl)pyrrolidin-3-one; 1-(3-(6-amino-2-fluoro-8-((7-(oxazol-2-yl)-2,3-dihydrobenzo[b][.1,4]dioxin-6-yl)methyl)-9H-purin-9-yl)propyl)pyTrolidin-3-one; and 9-(3-aminopropyl)-2-fluoro-8-((7-(5-metl yloxazol-2-yl)-2,3-dihydrobenzo[b][1,4]dioxin-6-yl)methyl)-9H-purin-6-amine.

In some embodiments of Formula VII, X2 is alkynyl.

In some embodiments, the epichaperome inhibitor is selected from the group consisting of: 8-((7-ethynyl-2,3-dihydrobenzo[b][1,4]dioxin-6-yl)thio)-9-(3-(isopropylamino)propyl)-9H-purin-6-amine; 3-(3-(6-amino-8-(7-ethynyl-2,3-dihydrobenzo[b][1,4]dioxin-6-ylthio)-9H-purin-9-yl)propyl)pyrrolidine-1-carbaldehyde; 8-((7-ethynyl-2,3-dihydrobenzo[b][1,4]dioxin-6-yl)thio)-9-(2-(neopentylamino)ethyl)-9H-purin-6-amine; 9-(2-aminoethyl)-8-((7-ethynyl-2,3-dihydrobenzo[b][1,4]dioxin-6-yl)thio)-9H-purin-6-amine; 1-(3-(2-(6-amino-8-(7-ethynyl-2,3-dihydrobenzo[b][1,4]dioxin-6-ylthio)-9H-purin-9-yl)ethyl)piperidin-1-yl)ethanone; 8-(7-ethynyl-2,3-dihydrobenzo[b][1,4]dioxin-6-ylthio)-9-(2-(1-(methylsulfonyl)piperidin-3-yl)ethyl)-9H-purin-6-amine; N-(2-((2-(6-amino-8-((7-ethynyl-2,3-dihydrobenzo[b][1,4]dioxin-6-yl)thio)-9H-purin-9-yl)ethyl)amino)ethyl)sulfamide; 9-(3-aminopropyl)-8-((7-ethynyl-2,3-dihydrobenzo[b][1,4]dioxin-6-yl)thio)-9H-purin-6-amine; 6-(6-amino-8-(7-ethynyl-2,3-dihydrobenzo[b][1,4]dioxin-6-ylthio)-9H-purin-9-yl)hexanamide; 5-(6-amino-8-(7-ethynyl-2,3-dihydrobenzo[b][1,4]dioxin-6-ylthio)-9H-purin-9-yl)pentane-1-sulfonamide; 1-(6-amino-8-((7-ethynyl-2,3-dihydrobenzo[b][1,4]dioxin-6-yl)thio)-9H-purin-9-yl)-3-(isopropylamino)propan-2-ol; 9-(3-(tert-butylamino)propyl)-8-(7-ethynyl-2,3-dihydrobenzo[b][1,4]dioxin-6-ylthio)-9H-purin-6-amine; 8-(7-ethynyl-2,3-dihydrobenzo[b]i1,4]dioxin-6-ylthio)-9-(2-(1-methylpiperidin-2-yl)ethyl)-9H-purm-6-amine; 8-(7-ethynyl-2,3-dihydrobenzo[b][1,4]dioxin-6-ylthio)-9-(2-(1-methylpiperidin-3-yl)ethyl)-9H-purin-6-amine; 9-(2-aminoethyl)-8-(7-ethynyl-2,3-dihydrobenzo[b][1,4]dioxin-6-ylthio)-9H-purin-6-amine; 8-((7-ethynyl-2,3-dihydrobenzo[b][1,4]dioxin-6-yl)methyl)-2-fluoro-9-(2-(isobutylamino)ethyl)-9H-purin-6-amine; 8-((7-ethynyl-2,3-dihydrobenzo[b][1,4]dioxin-6-yl)methyl)-2-fluoro-9-(2-(1-(methylsulfonyl)piperidin-3-yl)ethyl)-9H-purin-6-amine; 1-(3-(2-(6-amino-8-((7-ethynyl-2,3-dihydrobenzo[b][1,4]dioxin-6-yl)methyl)-2-fluoro-9H-purin-9-yl)ethyl)piperidin-1-yl)ethanone; 3-(2-(6-amino-8-((7-ethynyl-2,3-dihydrobenzo[b][1,4]dioxin-6-yl)memyl)-2-fluoro-9H-purin-9-yl)ethyI)piperidine-1-carbaldchyde; 1-(3-(6-amino-8-((7-ethynyl-2,3-dihydrobenzo[b][1,4]dioxin-6-yl)methyl)-2-fluoro-9H-purin-9-yl)propyl)pyrrolidin-3-one; 6-(6-amino-8-((7-ethynyl-2,3-dihydrobenzo[b][1,4]dioxin-6-yl)methyl)-2-fluoro-9H-purin-9-yl)hexanamide; 1-(6-amino-8-((7-ethynyl-2,3-dihydrobenzo[b][1,4]dioxin-6-yl)methyl)-2-flaoro-9H-purin-9-yl)-3-(tert\hat{ }butylamino)propan-2-ol; 5-(6-amino-8-((7-ethynyl-2J3-dihydrobenzo[b][1,4]dioxin-6-yl)methyl)-2-fluoro-9H-purin-9-yl)pentane-1-sulfonamide; 8-((7-ethynyl-2,3-dihydrobenzo[b][1,4]dioxm-6-yl)methyl)-2-fl\hat{ }\hat{ } amine; 9-(3-(tert-butylamino)propyl)-8-((7-ethynyl-2,3-dihydrobenzo[b][1,4]dioxin-6-yl)methyl)-2-fluoro-9H-purin-6-amine; 9-(3-aminopropyl)-8-((7-ethynyl-2,3-dihydrobenzo[b][1,4]dioxin-6-yl)methyl)-2-fluoro-9H-purin-6-amine; 8-((7-ethynyl-2,3-dihydrobenzo[b][1,4]dioxin-6-yl)methyl)-2-fluoro-9-(2-(1-methylpiperidin-2-yl)ethyl)-9H-purin-6-amine; and 8-((7-ethynyl-2,3-dihydrobenzo[b][1,4]dioxin-6-yl)methyl)-2-fluoro-9-(2-(1-methylpiperidin-3-yl)ethyl)-9H-purin-6-amine.

Another class of epichaperome inhibitors of this disclosure have the general structure of Formula VIII:

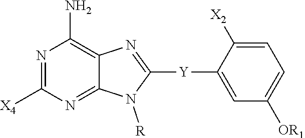

wherein

(a) R1 is alkyl;

(b) Y is S or CH2,

(c) X4 is H or halogen,

(d) X2 is a saturated or unsaturated non-aromatic carbocycle or heterocycle, an aryl, an alkylamino, a dialkylamino, an alkynyl or is part of a ring formed by R; and

(e) R is hydrogen, alkyl, alkenyl, or alkynyl, linear, branched or cyclic, optionally including heteroatoms such as N, S or O, optionally connected to the 2′-position to form an 8 to 10 member ring.

Other classes of epichaperome inhibitors of this disclosure have the general structure of Formulae IX, X or XI:

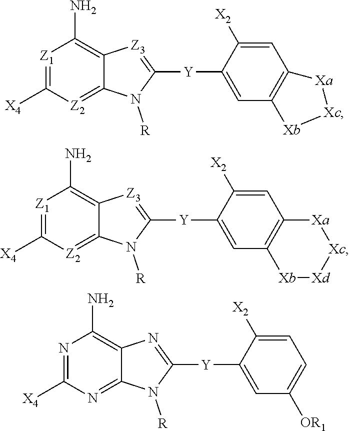

wherein

(a) Y is CH2, S, O, C═O, C═S, or N;

(b) Xd is H or halogen;

(c) Xa, Xb, Xc and Xd are independently selected from C, O, N, S, carbonyl, and thionyl, connected by single or double bonds with H as needed to satisfy valence,

(d) X2 is an alkynyl group and

(e) R is a group listed in Table A.

Other classes of epichaperome inhibitors of this disclosure have the general structure of Formulae XII, XIII or XIV:

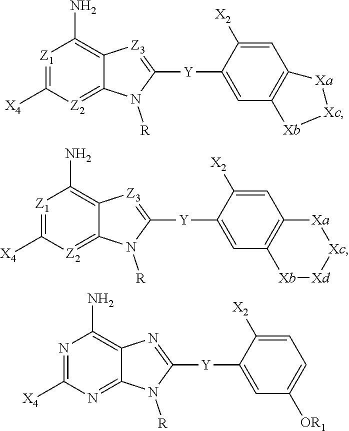

wherein

(a) Y is CH2, S, O, C═O, OS, or N; (b) X4 is H or halogen;

(c) Xa, Xb, Xc and Xd are independently selected from C, O, N, S, carbonyl, and thionyl, connected by single or double bonds with H as needed to satisfy valence,

(d) X2 is a furan, thiophene, pyrazole, oxazole or thiazole and

(e) R is a group listed in Table A.

TABLE A
R groups for Formulae VI-XIV

1. R is hydrogen, a C₁ to C₁₀ Alkyl, alkenyl, alkynyl, or an alkoxyalkyl group,
optionally including heteroatorns such as N or O, or a targeting moiety connected to N9 via a
linker,
2. R is hydrogen, straight- or branched-, substituted or unsubstituted alkyl,
substituted or unsubstituted alkenyl, substituted or unsubstituted alkynyl, in which one or
more methylenes can be interrupted or terminated by O, S, S(O), SO₂, N(R₂₁₈), C(O),
substituted or unsubstituted aryl, substituted or unsubstituted heteroaryl, substituted or
unsubstituted heterocyclic; substituted or unsubstituted cycloalkyl; or

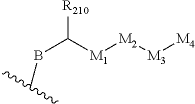

wherein
B is a linker;
R₂₁₀ is selected from the group consisting of hydrogen, N(R₂)COR₄,
N(R₂CON(R₃)R₄, N(R₂)COOR₄, M(R₂S(On)R₃, N(R₂)S(O)nN(R₃)R₄; where R₂ and R₃
are independently selected from hydrogen, aliphatic or substituted aliphatic; R₄ is selected
from the group consisting of: aryl, substituted aryl, heteroaryl, substituted heteroaryl,
heterocyclic, substituted heterocyclic, cycloalkyl, substituted cycloalkyl, cycloalkenyl,
substituted cycloalkenyl, and substituted or unsubstituted —Ci-C₆ alkyl, —C₂-C₆ alkenyl, or
—C₂-C₆ alkynyl each containing 0, 1, 2, or 3 heteroatoms selected from O, S or N; n is 1 or 2;
M1 is absent or selected from substituted or unsubstituted —Ci-C₆ alkyl,
—C₂-C₆ alkenyl, or —C₂-C₆ alkynyl, aryl, substituted aryl heteroaryl, substituted heteroaryl;
M2 is absent, O, S, SO, SO₂, N(R₂) or CO;
M3 is absent, O, S, SO, SO₂, N(R₂), CO, Ci-C₆ alkyl, C₂-C₆ alkenyl, C₂-C₆ alkynyl,
cycloalkyl, heterocyclic, aryl, or heteroaryl;
M4 is hydrogen, NR₅R₆, CF₃, OR₄, halogen, substituted or unsubstituted —C₁C₆ alkyl,
—C₂-C₆ alkenyl, or —C₂-C₆ alkynyl, cycloalkyl, substituted cycloalkyl, heterocyclic,
substituted heterocyclic, aryl, substituted aryl, heteroaryl or substituted heteroaryl; where
R₅ and R₆ are independently selected from the group consisting of hydrogen, aliphatic,
substituted aliphatic, aryl, substituted aryl, heteroaryl, substituted heteroaryl, heterocyclic,
substituted heterocyclic, cycloalkyl or substituted cycloalkyl; provided that —R and
—Mi—M₂—M₃—M₄ cannot be both hydrogen.
3. R is

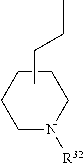

wherein R³² is
(a) hydro;
(b) C₁-C₆ alkyl optionally substituted with 1, 2, 3, 4, or 5 substituents each
independently chosen from the group of halo, hydroxyl, amino, cyano, and —C(═O)R³¹
wherein R³¹ is amino;
(c) —C(═Q)R³³, wherein R³³ is selected from the group consisting of:
(1) hydro,
(2) C₁C₁₀ (e.g., C₁-C₆) alkyl optionally substituted with 1, 2, 3, 4, or 5
substituents each independently chosen from the group of (A) halo, (B)
hydroxyl, (C) thiol, (D) cyano, (E) C₁-C₆ haloalkyl (e.g., trifluoromethyl), (F)
C₁-C₆ alkoxy (e.g., methoxy) optionally substituted with C₁-C₆ alkoxy (e.g.,
methoxy), (G) C-amido, (H) N-amido, (I) sulfonyl, (J) —N(R²²)(R²³) wherein
R²² and R²³ are independently hydro, C₁C₆ alkyl, sulfonyl, and C-carboxy,
(3) C₁-C₆ cycloalkyl optionally substituted with 1, 2, 3, 4, or 5
substituents each independently chosen from the group of halo, hydroxyl,
amino, cyano, and C₁-C₆ haloalkyl (e.g., trifluoromethyl), and
(4) C₁-C₆ alkoxy optionally substituted with 1, 2, 3, 4, or 5
substituents each independently chosen from halo, hydroxyl, amino, cyano,
and C₁-C₆ haloalkyl (e.g., trifluoromethyl),
(f) heterocycle or heterocyclylalkyl, optionally substituted with 1, 2, 3, 4, or 5
substituents independently chosen from halo, hydroxyl, amino, cyano, trihalomethyl, and
C₁-C₄ alkyl optionally substituted with 1, 2, 3, or 4 substituents independently chosen
from halo, hydroxyl, amino, cyano, C₁-C₆ haloalkyl (e.g., trifluoromethyl) (e.g., tetrazole-
5-yl optionally substituted with 1, 2, 3, or 4 C₁-C₄ alkyl);
(g) sulfonyl; and
(h) optionally substituted heteroaryl
4. R is —R⁵⁴—R⁵, wherein
R⁵⁴ is —(CH₂)n— wherein n = 0-3, —C(O), —C(S), —SO₂—, or —SO₂N—; and
R⁵⁵ is alkyl, aromatic, heteroaromatic, alicyclic, or heterocyclic, each of which is
optionally bi- or tri-cyclic, and optionally substituted with H, halogen, lower alkyl, lower
alkenyl, lower alkynyl, lower aryl, lower alicyclic, aralkyl, aryloxyalkyl, alkoxyalkyl,
perhaloalkyl, perhaloalkyloxy, perhaloacyl, —N₃, —SR⁵⁸, —OR⁵⁸, —CN, —CO₂R⁵⁹, —NO₂, or
—NR⁵⁸R⁵^{₁}⁰,
R⁵⁸ is hydrogen, lower alkyl, lower aryl, or —C(O)R5′5;
R⁵⁹ is lower alkyl, lower aryl, lower heteroaryl, —NR⁵^{₁}⁰R⁵^{₁}⁰ or —OR⁵^{₁₁};
R⁵^{₁}⁰ is independently hydrogen or lower alkyl; and
R⁵^{₁₁} is ______
5. R is selected from the group consisting of H, optionally substituted alkyl, optionally
substituted alkenyl, optionally substituted alkynyl, optionally substituted aryl, optionally
substituted alicyclic, optionally substituted araalkyl, optionally substituted aryloxyalkyl,
optionally substituted alkoxyalkyl, alkylaminoalkyl, alkylcarbonylaminoalkyl,
alkylcarbonyoxylalkyl, optionally substituted heterocyclic, hydroxyalkyl, haloalkyl,
perhaloalkyl, C(O)R⁶^{₂}, S(O)R⁶^{₂}, C(O)NHR⁶^{₂}, and C(O)OR⁶^{₂}; where R⁶^{₂}is
______
6. R is H, SR₇₁, SOR₇₁, SO₂R₇₁, OR₇₁, COOR₇₁, CONR₇₁R₇₂, —CN, C₁₋₆ alkyl, C₂₋₆
alkenyl, C₂₋₆ alkynyl, —R₇AOR₇B— —R₇AR₇B, —R₇ANR₇₁R₇B, —R₇ASR₇B,
—R₇ASOR₇B or —R₇ASO₂R₇B, cycloalkyl, heteroalkyl, heterocycloalkyl, aryl, heteroaryl,
alkylaryl, arylalkyl, alkylheteroaryl, heteroarylalkyl, NR₇₁R₇₂, —OSO₂N(R₇C₂,
—N(R₇C)SO₂OH, —N(R₇C)SO₂R₇C, —R₇AOSO₂N(R₇C)2, or —R₇AN(R₇C)OSO₂R₇C;
R₇₁ and R₇₂ are independently selected from the group consisting of H, COOR₇B,
CON(R₇C)₂ C₁₋₆ alkyl, C₂₋₆ alkenyl, C₂₋₆ alkynyl, —R₇AOR₇B~, —R₇ANR₇B,
—R₇ANR₇₁R₇B, —R₇ASR₇B, —R₇ASQR₇B or —R₇ASO₂R₇B cycloalkyl, heteroalkyl,
heterocycloalkyl, aryl, heteroaryl, alkylaryl, arylalkyl, alkylheteroaryl, and heteroarylalkyl;
each R₇A is independently C₁₋₆ alkyl, C₂₋₆ alkenyl, C₂₋₆ alkynyl, cycloalkyl,
heteroalkyl, heterocycloalkyl, aryl, heteroaryl, alkylaryl, arylalkyl, alkylheteroaryl,
alkylheteroarylalkyl, or heteroarylalkyl; and
each R₇B is independently H, C₁₋₆ alkyl, C₂₋₆ aLkenyl, C₂₋₆ alkynyl, cycloalkyl,
heteroalkyl, heterocycloalkyl, aryl, heteroaryl, alkylaryl, arylalkyl, alkylheteroaryl,
heteroarylalkyl, —SO₂OH—SO₂N(R₇A)₂, —SO₂NHR₇A or —SO₂NH₂; and
each R.sub.C is independently H, C₁₋₆ alkyl, C₂₋₆ alkenyl, C₂₋₆ alkynyl, cycloalkyl,
heteroalkyl, heterocycloalkyl, aryl, heteroaryl, alkylaryl, arylalkyl, alkylheteroaryl, or
heteroarylalkyl;
7A. R is hydrogen, straight- or branched-, substituted or unsubstituted alkyl, substituted
or unsubstituted alkenyl, substituted or unsubstituted alkynyl, which one or more
methylenes can be interrupted or terminated by O, S, S(O), SO₂, N(R₈₈), C(O), substituted
or unsubstituted aryl, substituted or unsubstituted heteroaryl, substituted or unsubstituted
heterocyclic; substituted or unsubstituted cycloalkyl; where R₈₈ is hydrogen, acyl, aliphatic
or substituted aliphatic,
7B. R is —M1—M2—M3—M4, wherein
M₁ is absent, C₁-C₆ alkyl, C₂-C₆ alkenyl, C₂-C₆ alkynyl, aryl or heteroaryl;
M₂ is absent, O, S, SO, SO₂, N(R₈₈), or C = O;
M₃ is absent, C = O, O, S, SO, SO₂ or N(R₈₈); and
M₄ is hydrogen, halogen, CN, N₃, hydroxy, substituted hydroxy, amino, substituted
amino, CF₃, C₁-C₆ alkyl, C₂-C₆ alkenyl, C₂-C₆ alkynyl, cycloalkyl, heterocyclic, aryl or
heteroaryl.

“Alkyl” (or alkyl group) refers to a linear, cyclic or branched saturated hydrocarbon, for example a hydrocarbon having from 1 to 10 carbon atoms, in which the atom directly attached to the central structure is a carbon atom. Such an alkyl group may include substituents other than hydrogen, for example an oxygen-containing group including without limitation hydroxyl and alkoxy; a halogen group; a nitrogen-containing group including without limitation amino, amido and alkylamino; an aryl group; a sulfur-containing group including without limitation thioalkyl; and/or a non-aromatic cyclic group including heterocycles and carbocycles. Carbon atoms in these substituents may increase the total number of carbon atoms in the alkyl group to above 10 without departing from the spirit of this disclosure. All references to alkyl groups in the specification and claims hereof encompass both substituted and unsubstituted alkyl groups unless the context is clearly to the contrary.

“Alkenyl” (or alkenyl group) refers to a linear, cyclic or branched hydrocarbon, for example a hydrocarbon having from 1 to 10 carbon atoms, and at least one double bond, in which the atom directly attached to the central structure is a carbon atom. The alkenyl group may include any of the substituents mentioned above for an alkyl group. All references to alkenyl groups in the specification and claims hereof encompass both substituted and unsubstituted alkenyl groups unless the context is clearly to the contrary.

“Alkynyl” (or alkynyl group) refers to a linear, cyclic or branched hydrocarbon, for example a hydrocarbon having from 1 to 10 carbon atoms, and at least one triple bond, in which the atom directly attached to the central structure is a carbon atom. The alkynyl group may include any of the substituents mentioned above for an alkyl group. All references to alkynyl groups in the specification and claims hereof encompass both substituted and unsubstituted alkynyl groups unless the context is clearly to the contrary.

“Aryl” (or aryl group) refers to any group derived from a simple aromatic ring. Aryl group includes heteroaryl. Aryl groups may be substituted or unsubstituted. When X2, X4 and R is identified as an aryl group (particularly for Formulae VI-XIV), an atom of the aryl ring is bound directly to an atom of the central structure. An aryloxy substituent is an aryl group connected to the central structure through an oxygen atom. The aryl group may include any of the substituents mentioned above for an alkyl group, and in addition an aryl group may include an alkyl, alkenyl or alkynyl group. All references to aryl groups in the specification and claims hereof encompass both substituted and unsubstituted aryl groups unless the context is clearly to the contrary.

“Amino” (or amino group) refers to any group which consists of a nitrogen attached by single bonds to carbon or hydrogen atoms. In certain instances, the nitrogen of the amino group is directly bound to the central structure. In other instances, an amino group may be a substituent on or within a group, with the nitrogen of the amino group being attached to the central structure through one or more intervening atoms. Examples of amino groups include NH2, alkylamino, alkenylamino groups and N-containing non-aromatic heterocyclic moiety (i.e., cyclic amines). Amino groups may be substituted or unsubstituted. All references to amino groups in the specification and claims hereof encompass substituted and unsubstituted amino groups unless the context is clearly to the contrary.

“Halogen” (or halogen group) refers to fluorine, chlorine, bromine or iodine.

“Heterocyclic” (or heterocyclic group) refers to a moiety containing at least one atom of carbon, and at least one atom of an element other than carbon, such as sulfur, oxygen or nitrogen within a ring structure. These heterocyclic groups may be either aromatic rings or saturated and unsaturated non-aromatic rings. Heterocyclic groups may be substituted or unsubstituted. All references to heterocyclic groups in the specification and claims encompass substituted and unsubstituted heterocyclic groups unless the context is clearly to the contrary.

In the compounds provided herein, all of the atoms have sufficient hydrogen or non-hydrogen substituents to satisfy valence, or the compound includes a pharmaceutically acceptable counterion, for example in the case of a quaternary amine.

Additional examples of compounds of this type are provided by in US published application US 2009/0298857 A1 and in U.S. Pat. No. 7,834,181, the entire disclosures of which as they relate to such Hsp90 inhibitors and classes thereof are incorporated by reference herein.

Reference can also be made to PCT Publication No. WO2011/044394 (Application No. PCT/US2010/051872) for additional compounds that can be used as Hsp90 inhibitors and that are contemplated as part of this disclosure. The teachings of such reference are incorporated by reference herein, particularly with respect to their disclosure of compounds of any one of Formulae VI-XIV (as named herein).

The methods and products provided herein may use or comprise a single BBB-permeable epichaperome inhibitor. Alternatively, the methods and products provided herein may comprise one or more epichaperome inhibitors, provided at least one is a BBB-permeable epichaperome inhibitor. In some embodiments, the BBB-permeable Hsp90 inhibitor is Compound 1.

The epichaperome inhibitors may be provided as pharmaceutically acceptable salts. The term “pharmaceutically acceptable salt” refers to those salts which retain the biological effectiveness and properties of the “free” compounds provided herein. A pharmaceutically acceptable salt can be obtained from the reaction of the free base of an active compound provided herein with an inorganic acid, for example, hydrochloric acid, hydrobromic acid, sulfuric acid, nitric acid, phosphoric acid, and the like, or an organic acid, for example, sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, tartaric acid (e.g., (+)-tartaric acid or (−)-tartaric acid or mixtures thereof), and the like.

Certain active compounds provided herein have acidic substituents and can exist as pharmaceutically acceptable salts with pharmaceutically acceptable bases. The present disclosure includes such salts. Examples of such salts include metal counterion salts, such as sodium, potassium, lithium, magnesium, calcium, iron, copper, zinc, silver, or aluminum salts, and organic amine salts, such as methylamine, dimethylamine, trimethylamine, diethylamine, triethylamine, n-propylamine, 2-propylamine, or dimethylisopropylamine salts, and the like.

The term “pharmaceutically acceptable salt” includes mono-salts and compounds in which a plurality of salts is present, e.g., di-salts and/or tri-salts. Pharmaceutically acceptable salts can be prepared by methods known to those in the art.

In some instances, the BBB-permeable epichaperome inhibitor is labelled with a moiety that can be detected using an imaging modality such as MRI, PET or SPECT. In preferred embodiments thereof, the labelling of the BBB-permeable epichaperome inhibitor with such moieties does not significantly impact its ability to traverse the BBB or its residence time in the brain.

Imaging agents for magnetic resonance imaging (MRI) include Gd(DOTA), iron oxide or gold nanoparticles; imaging agents for nuclear medicine include 201T1, gamma-emitting radionuclide 99 mTc; imaging agents for positron-emission tomography (PET) include positron-emitting isotopes such as ¹³¹I or 1²⁴I, (18)F-fluorodeoxyglucose ((18)FDG), (18)F-fluoride, copper-64, gadoamide, and radioisotopes of Pb(II) such as 203 Pb, and 11In.

In some embodiments, the epichaperome inhibitor is not detectably labelled. For example, it may not be labelled with a radioisotope.

### BBB-Permeability

The foregoing list of epichaperome inhibitors may be tested for BBB-permeability using techniques known in the art. For example, such agents may be labelled with a detectable marker such as but not limited to a radioisotope and their ability to cross the BBB (as indicated by brain uptake of the agent) may be determined by standard imaging techniques including but not limited to MRI, SPECT, PET, and the like. The agents may be radioactively labelled and autoradiography may be used to determine location and uptake of the agent. Typically the agents will be administered remotely from the brain, including for example intravenously, intramuscularly, and the like. It will be understood by those of ordinary skill in the art that the imaging modality will typically dictate the nature of the detectable marker (or detectable label, as the terms are used interchangeably herein). If done in experimental animals, the brains of such animals may be biopsied to determine the amount of agent present therein. The BBB-permeability of the agents may also be determined using in vitro techniques such culture of brain microvessels, endothelial cells, and the like, which may be freshly isolated, primary or cell lines. Reference can be made to Bickel, NeuroRx, 2005, 2(1):15-26. Reference may also be made to published PCT application WO2008/005937.

Putative epichaperome inhibitors may also be screened for their ability to act as a substrate for transport proteins that function to extrude agents from the brain. One such transport protein is P-glycoprotein. Thus, in some but not all instances, the BBB-permeable epichaperome inhibitors may also be characterized as not being substrates for P-glycoprotein or other transport proteins.

Brain uptake may be indicative of binding of the epichaperome inhibitor to Hsp90 and preferably to brain Hsp90. Such binding assays may be carried out as described in published PCT application WO2008/005937.

BBB-permeable epichaperome inhibitors to be used in the methods provided herein may be further characterized by their EC50 for binding to Hsp90 such as brain Hsp90. (EC50, in this context, refers to the concentration of the inhibitor that yields half-maximal binding of the inhibitor to Hsp90 such as brain Hsp90, or alternatively if the measurement is performed in a competition assay it is the concentration of inhibitor at which the binding to Hsp90 is reduced by half.) Certain inhibitors may have an EC50 of 100 nM or less, including about 90 nM, about 80 nM, about 85 nM, about 70 nM, about 60 nM, about 50 nM, about 40 nM, about 30 nM, about 20 nM, about 10 nM, about 9 nM, about 8 nM, about 7 nM, about 6 nM, about 5 nM, about 4 nM, about 3 nM, about 2 nM, or about 1 nM. The EC50 might range from 0.1 to 10 nM, from 0.1 to 9 nM, from 0.1 to 8 nM, from 0.1 to 7 nM, from 0.1 to 6 nM, or from 0.1 to 5 nM. The EC50 might range from 0.1 to 20 nM, from 0.1 to 18 nM, from 0.1 to 15 nM, from 0.1 to 12 nM, or from 0.1 to 10 nM. In some instances, certain inhibitors have an EC50 of about 5-7 nM. Compound 1, for example, has an EC50 of 6.9 nM. In other instances, certain inhibitors have an EC50 of about 80-90 nM. Compound 2 has an EC50 of 85.3 and 40.1 nM, as reported in published US application 2014/0378452.

### Traumatic Brain Injury (TBI)

Traumatic brain injury (TBI) as used herein refers to an injury resulting from external mechanical force applied to the brain that causes brain dysfunction. The external mechanical force may be referred to herein as the traumatic event. The traumatic event is typically a violent blow or jolt to the head or body that nevertheless results in brain dysfunction. Such brain dysfunction may be apparent immediately after the traumatic event or it may be apparent within hours or days of the traumatic event.

Traumatic events may occur as a result of a fall, participation in a high-risk sport such as football, hockey, soccer, rugby, boxing or other contact sport, involvement in a motor vehicle collision either as a passenger or pedestrian (by-stander), involvement in a bicycle collision either as a rider or a pedestrian (by-stander), involvement in combat (e.g., as a solider or by-stander), exposure to, including close proximity to, bomb blasts, physical abuse such as violent head shaking or blows to the head, skull penetration of an object such as a bullet or shrapnel or shattered skull, and the like.

TBI may be diagnosed by the presence of one or more TBI-related symptoms and/or by imaging of the brain, typically following the occurrence of a traumatic event. Such symptoms may first arise within a week to a few weeks or few months of the traumatic event. Similarly, these symptoms may persist for days, weeks, or months following the traumatic event.

TBI-related symptoms include headache or sensation of pressure in the head, temporary loss of consciousness, confusion, amnesia surrounding the traumatic event giving rise to the TBI, dizziness, ringing in the ears, nausea, vomiting, slurred speech, delayed responsiveness (e.g., delayed response to questions), appearing dazed, fatigue, pupil dilation, compromised vision, and difficulty breathing. One or more symptoms may arise immediately after the traumatic event, or they may arise within hours or even days of the traumatic event. Delayed symptoms may include, but are not limited to, concentration and memory deficiencies, irritability and/or other personality changes, sensitivity to light and/or sound, changes in sleep patterns, changes to ability to taste and/or smell, and psychological adjustment issues and depression.

TBI may be mild, moderate or severe, depending on the number, severity and duration of symptoms. Mild TBI is typically associated with temporary brain dysfunction. Severe TBI may be associate with bruising, torn tissues, bleeding and other physical damage to the brain. Certain TBI may be associated with concussions. Concussions typically refer to non-structural, typically non-haemorrhaging, injuries of the brain. Most concussions are not diagnosed using neuroimaging tests such as CT or MRI.

The methods provided herein are intended to provide therapeutic benefit to subjects that have experienced a TBI. Such therapeutic benefit may impact short term sequelae of the TBI and/or they may impact the long-term sequelae of the TBI. An example of a long-term sequelae of a TBI is believed to be chronic traumatic encephalopathy (CTE). CTE refers to a condition characterized by progressive brain deterioration and caused by one and typically repeated TBIs. An example of CTE is another condition referred to as dementia pugilistica (DP) which tends to be diagnosed in those with a history of boxing. Some regard it as a tauopathy. Hallmark symptoms associated with CTE usually manifest themselves several years after the occurrence of the TBIs. These symptoms include deterioration in attention as well as disorientation, dizziness and headaches. As the condition progresses, memory loss, social instability, erratic behaviour, and poor judgement are also apparent. The latter stages of the condition involve progressive dementia, slowing of muscular movements, impeded speech, tremors, vertigo, deafness, and suicidal tendencies. The methods provided herein which are geared towards early intervention following a TBI are expected to benefit TBI subjects both in the short-term as well as in the long-term including for example by reducing their risk of developing CTE, delaying the manifestation of CTE, and/or reducing the severity of CTE if and when it does develop. The short-term readouts, such as the short-term symptoms associated with TBI, in a sense may act as surrogates for the ability to impact the progression of CTE in such subjects.

### Additional Therapies

The methods provided herein contemplate treating subjects that have experienced a TBI with a BBB-permeable Hsp90 inhibitor, optionally along one or more other therapies. Such secondary therapies may be chemical therapies such as administration of secondary therapeutic agents (e.g., anti-inflammatories and/or analgesic agents), or they may be non-chemical therapies. An example of this latter type of therapy includes general immobilization of the subject, as may be achieved through bed rest for example. The Hsp90 inhibitors and secondary therapeutic agents may have an additive therapeutic effect or a synergistic (i.e., greater than additive) therapeutic effect on the subject.

An anti-inflammatory agent is an agent that reduces inflammation in a subject. Certain anti-inflammatory agents also act as analgesics (i.e., as pain-reducing agents). Such agents may be referred to as dual-acting agents herein. Examples of anti-inflammatory agents, some of which are dual-acting agents, include but are not limited to such as non-steroidal anti-inflammatory drugs (NSAIDs, such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, indomethacin, COX1/COX2 inhibitors, anti-TNF-.alpha. compounds, infliximab, etanercept, adalimumab, and the like.

In some embodiments, the anti-inflammatory agent can be a steroid (e.g., a corticosteroid or glucocorticoid); a calcineurin inhibitor (e.g. cyclosporine, tacrolimus, pimecrolimus, or FK506); an mTOR inhibitor (e.g., everolimus, temsirolimus, rapamycin, deforolimus, TOP216, OSI-027, TAFA93, nab-rapamycin, tacrolimus, biolimus, CI-779, ABT-578, AP-23675, BEZ-235, QLT-0447, ABI-009, BC-210, salirasib, AP-23841, AP-23573, KU-0059475, 32-deoxorapamycin, 16-pent-2-ynyloxy-32-deoxorapamycin, 16-pent-2-ynyloxy-32 (S or R)-dihydro-rapamycin, 16-pent-2-ynyloxy-32 (S or R)-dihydro-40-O-(2-hydroxyethyl)-rapamycin, 40-O-(2-hydroxyethyl)-rapamycin, 32-deoxorapamycin; 16-pent-2-ynyloxy-32(S)-dihydrorapamycin; socalledrapalogs; AP23464; PI-103, PP242, PP30, Torinl; and derivatives or pharmaceutically acceptable salts thereof as well as and compounds described in, e.g. U.S. Patent Publications 2011/0178070; 2011/0021515; 2007/0112005; 2011/0054013; International Patent Publications WO98/02441; WO01/14387; WO99/15530; WO07/135411; WO03/64383; WO96/41807; WO95/16691; WO94/09010; European Patent No. EP1880723; and U.S. Pat. Nos. 8,163,775; 6,329,386; 6,200,985; 6,117,863; 6,015,815; 6,015,809; 6,004,973; 5,985,890; 5,955,457; 5,922,730; 5,912,253; 5,780,462; 5,665,772; 5,637,590; 5,567,709; 5,563,145; 5,559,122; 5,559,120; 5,559,119; 5,559,112; 5,550,133; 5,541,192; 5,541,191; 5,532,355; 5,530,121; 5,530,007; 5,525,610; 5,521,194; 5,519,031; 5,516,780; 5,508,399; 5,508,290; 5,508,286; 5,508,285; 5,504,291; 5,504,204; 5,491,231; 5,489,680; 5,489,595; 5,488,054; 5,486,524; 5,486,523; 5,486,522; 5,484,791; 5,484,790; 5,480,989; 5,480,988; 5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300; 5,260,299; 5,233,036; 5,221,740; 5,221,670; 5,202,332; 5,194,447; 5,177,203; 5,169,851; 5,164,399; 5,162,333; 5,151,413; 5,138,051; 5,130,307; 5,120,842; 5,120,727; 5,120,726; 5,120,725; 5,118,678; 5,118,677; 5,100,883; 5,023,264; 5,023,263; and 5,023,262; which are incorporated by reference herein in their entireties.); rapamycin (sirolimus) or an analogue thereof (e.g. everolimus, temsirolimus, ridaforolimus, deforolimus); or an anti-proliferative agent (e.g. mycophenoloate moefitil, azathioprine). In some embodiments, the mTOR inhibitor can be rapamycin or an analogue thereof, e.g. everolimus, temsirolimus, ridaforolimus, or deforolimus. Anti-proliferative agents can include, by way of non-limiting example, alkylating agents (e.g. cyclophosphamide, platinum compounds, and nitrosoureas), antimetabolites (e.g. methotrexate, azathioprine, mercaptopurine, fluorouracil, etc), and cytotoxic antibiotics (e.g., dactinomycin, anthracyclines, mitomycin C, bleomycin, and mithramycin).

Examples of secondary therapeutic agents include angiogenesis inhibitors, pro-apoptotic agents, cell cycle arrest agents, kinase inhibitors, AKT inhibitors, BTK inhibitors, Bcl2 inhibitors, SYK inhibitors, CD40 inhibitors, CD28 pathway inhibitors, MHC class II inhibitors, PI3K inhibitors, mTOR inhibitors, JAK inhibitors, IKK inhibitors, Raf inhibitors, SRC inhibitors, phosphodiesterase inhibitors, ERK-MAPK pathway inhibitors, and the like.

Examples of AKT inhibitors include PF-04691502, Triciribine phosphate (NSC-280594), A-674563, CCT128930, AT7867, PHT-427, GSK690693, MK-2206 dihydrochloride.

Examples of BTK inhibitors include PCI-32765.

Examples of Bcl2 inhibitors include ABT-737, Obatoclax (GX15-070), ABT-263.
TW-37 Examples of SYK inhibitors include R-406, R406, R935788 (Fostamatinib disodium).

Examples of CD40 inhibitors include SGN-40 (anti-huCD40 mAb).

Examples of inhibitors of the CD28 pathway include abatacept, belatacept, blinatumomab, muromonab-CD3, visilizumab.

Examples of inhibitors of major histocompatibility complex, class II include apolizumab.

Examples of PI3K inhibitors include 2-(1H-indazol-4-yl)-6-(4-methanesulfonylpiperazin-1-ylmethyl)-4-morpholin-4-ylthieno(3,2-d)pyrimidine, BKM120, NVP-BEZ235, PX-866, SF 1126, XL147.

Example of mTOR inhibitors include deforolimus, everolimus, NVP-BEZ235, OSI-027, tacrolimus, temsirolimus, Ku-0063794, WYE-354, PP242, OSI-027, GSK2126458, WAY-600, WYE-125132.

Examples of JAK inhibitors include Tofacitinib citrate (CP-690550), AT9283, AG-490, INCBO 18424 (Ruxolitinib), AZD1480, LY2784544, NVP-BSK805, TGI 01209, TG-101348.

Examples of IkK inhibitors include SC-514, PF 184.

Examples of inhibitors of Raf include sorafenib, vemurafenib, GDC-0879, PLX-4720, PLX4032 (Vemura/enib), NVP-BHG712, SB590885, AZ628, ZM 336372.

Examples of inhibitors of SRC include AZM-475271, dasatinib, saracatinib.

Examples of inhibitors of phosphodiesterases include aminophylline, anagrelide, arofylline, caffeine, cilomilast, dipyridamole, dyphylline, L 869298, L-826,141, milrinone, nitroglycerin, pentoxifylline, roflumilast, rolipram, tetomilast, theophylline, tolbutamide, amrinone, anagrelide, arofylline, caffeine, cilomilast, L 869298, L-826,141, milrinone, pentoxifylline, roflumilast, rolipram, tetomilast.

Other secondary therapeutic agents that can be used in combination with the Hsp90 inhibitors of this disclosure include AQ4N, becatecarin, BN 80927, CPI-0004Na, daunorubicin, dexrazoxane, doxorubicin, elsamitrucin, epirubicin, etoposide, gatifloxacin, gemifloxacin, mitoxantrone, nalidixic acid, nemorubicin, norfloxacin, novobiocin, pixantrone, tafluposide, TAS-103, tirapazamine, valrubicin, XK469, BI2536.

Still other secondary therapeutic agents are nucleoside analogs. Examples include (1) deoxyadenosine analogues such as didanosine (ddI) and vidarabine; (2) adenosine analogues such as BCX4430; (3) deoxycytidine analogues such as cytarabine, gemcitabine, emtricitabine (FTC), lamivudine (3TC), and zalcitabine (ddC); (4) guanosine and deoxyguanosine analogues such as abacavir, acyclovir, and entecavir; (5) thymidine and deoxythymidine analogues such as stavudine (d4T), telbivudine, zidovudine (azidothymidine, or AZT); and (6) deoxyuridine analogues such as idoxuridine and trifluridine.

Other secondary therapeutic agents include taxanes such as paclitaxel, dicetaxel, cabazitaxel. Other secondary therapeutic agents include inhibitors of other heatshock proteins such as of Hsp70, Hsp60, and Hsp26.

Still other secondary therapeutic agents that can be used in combination with the epichaperome inhibitors of this disclosure are disclosed in published PCT Application No. WO2012/149493, the entire disclosure of which as it relates to such secondary therapeutic agents and classes thereof is incorporated by reference herein.

The epichaperome inhibitors and the secondary therapeutic agents may be co-administered. Co-administered includes administering substantially simultaneously, concomitantly, sequentially or adjunctively. The epichaperome inhibitors and the secondary therapeutic agents may be administered at different times and through different routes. For example, the BBB-permeable epichaperome inhibitors may be administered before or after the secondary therapeutic agent including one or more hours before, one or more day before, or one or more week before the secondary therapeutic agents. One or more secondary therapeutic agents may be used. Each of the therapeutic agents may be administered at their predetermined optimal frequency and dose. In some instances, the BBB-permeable epichaperome inhibitors and the secondary therapeutic agents are administered in combination in a therapeutically effective amount.

### Formulations Generally

The agents described herein, including the Hsp90 inhibitors and importantly the BBB-permeable epichaperome inhibitors, may be formulated for a variety of administration routes including without limitation oral delivery, intranasal delivery, delivery by inhalation, parenteral delivery, and the like. Preferably, the administration route is amenable to the status of the subject being treated. Thus, if the subject is unconscious or is having trouble swallowing, then a formulation that dissolves in the mouth (e.g., under the tongue on in the cheek area) may be preferred, as may be intranasal formulation (e.g., a spray to be administered in the nose) or an inhaled formulation that may be administered with an inhaler or a nebulizer, or a parenteral formulation that may be injected intramuscularly, for example,

Oral Formulations Generally

The oral formulation may take any one of a variety of solid, semi-solid or liquid forms. Examples of solid forms include without limitation coated or uncoated capsules or tablets, immediate release or altered release capsules or tablets including extended-release and delayed release capsules or tablets, as well as controlled release capsules or tablets. Such oral formulations may further comprise one or more excipients such as but not limited to anti-adherents, binders, fillers, lubricants, glidants, disintegrating agents, dispersion agents, solubilizing agents, sweetening or flavouring agents, surfactants. Liquid forms may be solutions, suspensions, emulsions, syrups and the like. Excipients that may be used in oral liquids include but are not limited to buffering agents (i.e., buffers), coloring agents, flavoring agents, sweetening agents, preservatives, anti-oxidants, and suspending agents.

One example of a suitable oral formulation is a disintegrating tablet formulation. A disintegrating tablet is an alternative to conventional tablets or capsules. One advantage of disintegrating tablets is improved patient compliance particularly in patients who have difficulty swallowing tablets and capsules generally. Disintegrating tablets are tablets that disintegrate in the oral cavity (mouth). Such tablets may comprise one or more, including two, three, four, five or more categories of excipients selected from the group consisting of filler/diluent, binder, lubricant, glidant, disintegrating agent, sweetening or flavouring agent, and/or dispersion agent.

In some exemplary formulations, the oral disintegrating tablets are formulated with 10 mg and 50 mg of API per tablet. There are six excipients in each tablet. An example of the composition of each dosage strength oral disintegrating tablet is provided below.

### Composition of Oral Disintegrating Tablet

| | Amount per Dosage Strength | |
|---|---|---|
| Component | 10 mg | 50 mg |
| Compound 1 (epichaperome inhibitor) | 10 mg | 50 mg |
| F-Melt | 200 mg | 200 mg |
| Crospovidone | 8.0 mg | 8.0 mg |
| (disintegrant, also known as | | |
| Polyvinylpolypyrrolidone (polyvinyl | | |
| polypyrrolidone, PVPP) | | |
| Sucralose | 3.0 mg | 3.0 mg |
| (sweetener) | | |
| Sodium stearyl fumarate | 3.0 mg | 3.0 mg |
| (lubricant) | | |
| Strawberry flavor | 0.7 mg | 0.7 mg |
| Masking flavor | 0.3 mg | 0.3 mg |
| (flavoring agent and taste masking | | |
| agent) | | |
| Target tablet weight (mg) | 225 mg | 265 mg |

### Subjects

The subjects to be treated and for whom the methods and products provided herein are intended include mammals such as humans and animals such as non-human primates, agricultural animals (e.g., cow, pig, sheep, goat, horse, rabbit, etc.), companion animals (e.g., dog, cat, etc.), and rodents (e.g., rat, mouse, etc.). Preferred subjects are human subjects. Subjects may be referred to herein as patients in some instances.

In some embodiments, the subject does not have a neurodegenerative disease such as but not limited to Alzheimer's disease and tauopathy, nor does the subject have CTE. Thus, in some instances, the subject may present with symptoms associated with a concussion and such symptoms may have been present only since the TBI.

The subject is typically less than 75 years old, more typically less than 60 years old, and even more typically less than 50 years old. The subject may be less than 45 years old, less than 40 years old, less than 35 years old, less than 30 years old, less than 25 years old, less than 20 years old, less than 15 years old, or less than 10 years old. Humans typically present with neurodegenerative disease at an older age and thus the age of the subjects to be treated according to this disclosure is typically younger than the typical age of onset of neurodegenerative disease.

### Kits

This disclosure further provides kits comprising the BBB-permeable Hsp90 inhibitors together with instructions for use in treating a subject that has experienced or is experiencing a TBI.

The kits may comprise any of the formulations discussed herein including oral formulations, inhaled formulations, intranasal formulations, and parenteral formulations such as injectable formulations. Oral formulations such as tablets or capsules may be packaged (or housed) with a fluid such as water for ingestion, a straw, a cup, a bottle, etc. Any of these formulations may be provided in a concentrated form and with instructions for diluting the formulation prior to administration. Intranasal formulations may be provided with or in bottles such as spray bottles. Inhaled formulations may be provided in ampoules, with or without a nebulizer. Injectable formulations may be provided with or in a syringe (e.g., a prefilled syringe) or with or in an auto-injection device (typically for intramuscular injection).

### OTHER EMBODIMENTS AND EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method for treating a subject that has experienced traumatic brain injury (TBI) comprising
administering to the subject an effective amount of a BBB-permeable epichaperome inhibitor within 2 weeks of the TBI.

2. The method of claim 1, wherein the TBI is a mild TBI.

3. The method of claim 1, wherein the TBI is a moderate TBI.

4. The method of claim 1, wherein the TBI is a severe TBI.

5. The method of any one of claims 1-4, wherein the BBB-permeable epichaperome inhibitor is administered within 5 days of the TBI.

6. The method of any one of claims 1-4, wherein the BBB-permeable epichaperome inhibitor is administered within 2 or 4 hours of the TBI.

7. The method of any one of claims 1-6, wherein the BBB-permeable epichaperome inhibitor is administered repeatedly.

8. The method of any one of claims 1-6, wherein the BBB-permeable epichaperome inhibitor is administered twice a day, three times a day, or four times a day, for 1 day or more.

9. The method of any one of claims 1-8, wherein the BBB-permeable epichaperome inhibitor is administered orally.

10. The method of claim 9, wherein the BBB-permeable epichaperome inhibitor is formulated as a capsule, tablet, lozenge, sublingual formulation, solution or suspension.

11. The method of any one of claims 1-8, wherein the BBB-permeable epichaperome inhibitor is administered parenterally.

12. The method of any one of claims 1-8, wherein the BBB-permeable epichaperome inhibitor is administered intramuscularly.

13. The method of any one of claims 1-8, wherein the BBB-permeable epichaperome inhibitor is administered using an auto-injector.

14. The method of any one of claims 1-8, wherein the BBB-permeable epichaperome inhibitor is administered intranasally or by inhalation.

15. The method of any one of claims 1-8, wherein the BBB-permeable epichaperome inhibitor is administered using an inhaler or a nebulizer.

16. The method of any one of claims 1-15, wherein the BBB-permeable epichaperome inhibitor is a compound having a structure of Formula I, or Formula II, or Formula III, or Formula IV, or Formula V, or Formula VIa, or Formula VIb.

17. The method of any one of claims 1-15, wherein the BBB-permeable epichaperome inhibitor is Compound 1.

18. The method of any one of claims 1-17, further comprising administering a second therapeutic agent to the subject.

19. The method of claim 18, wherein the BBB-permeable epichaperome inhibitor and the second therapeutic agent are administered simultaneously.

20. The method of claim 18, wherein the BBB-permeable epichaperome inhibitor and the second therapeutic agent are administered in an alternating manner.

21. The method of any one of claims 18-20, wherein the second therapeutic agent is an anti-inflammatory agent.

22. The method of any one of claims 18-20, wherein the second therapeutic agent is an analgesic.

23. A method for reducing sequelae of traumatic brain injury (TBI) comprising
administering, to a subject that has experienced a TBI, an effective amount of a BBB-permeable epichaperome inhibitor.

24. The method of claim 23, wherein reducing sequelae comprises reducing number of sequelae, reducing severity of one or more sequelae, reducing duration of one or more sequelae, and/or delaying onset of one of more sequelae.

25. The method of claim 23 or 24, wherein the TBI is a mild TBI.

26. The method of claim 23 or 24, wherein the TBI is a moderate TBI.

27. The method of claim 23 or 24, wherein the TBI is a severe TBI.

28. The method of any one of claims 23-27, wherein the BBB-permeable epichaperome inhibitor is administered within 2 weeks or with 8 days of the TBI.

29. The method of any one of claims 23-27, wherein the BBB-permeable epichaperome inhibitor is administered within 2 or 4 hours of the TBI.

30. The method of any one of claims 23-29, wherein the BBB-permeable epichaperome inhibitor is administered repeatedly.

31. The method of any one of claims 23-29, wherein the BBB-permeable epichaperome inhibitor is administered twice a day, three times a day, or four times a day, for 1 day or more.

32. The method of any one of claims 23-31, wherein the BBB-permeable epichaperome inhibitor is administered orally.

13. The method of claim 32, wherein the BBB-permeable epichaperome inhibitor is formulated as a capsule, tablet, lozenge, sublingual formulation, solution or suspension.

34. The method of any one of claims 23-31, wherein the BBB-permeable epichaperome inhibitor is administered parenterally.

35. The method of any one of claims 23-31, wherein the BBB-permeable epichaperome inhibitor is administered intramuscularly.

36. The method of any one of claims 23-31, wherein the BBB-permeable epichaperome inhibitor is administered using an auto-injector.

37. The method of any one of claims 23-31, wherein the BBB-permeable epichaperome inhibitor is administered intranasally or by inhalation.

38. The method of any one of claims 23-31, wherein the BBB-permeable epichaperome inhibitor is administered using an inhaler or a nebulizer.

39. The method of any one of claims 23-38, wherein the BBB-permeable epichaperome inhibitor is a compound having a structure of Formula I, or Formula II, or Formula III, or Formula IV, or Formula V, or Formula VIa, or Formula VIb.

40. The method of any one of claims 23-38, wherein the BBB-permeable epichaperome inhibitor is Compound 1.

41. The method of any one of claims 23-40, further comprising administering a second therapeutic agent to the subject.

42. The method of claim 41, wherein the BBB-permeable epichaperome inhibitor and the second therapeutic agent are administered simultaneously.

43. The method of claim 41, wherein the BBB-permeable epichaperome inhibitor and the second therapeutic agent are administered in an alternating manner.

44. The method of any one of claims 41-43, wherein the second therapeutic agent is an anti-inflammatory agent.

45. The method of any one of claims 41-43, wherein the second therapeutic agent is an analgesic.

46. The method of any one of claims 24-45, wherein the one or more sequelae are selected from headache or sensation of pressure in the head, temporary loss of consciousness, confusion, amnesia surrounding the traumatic event giving rise to the TBI, dizziness, ringing in the ears, nausea, vomiting, slurred speech, delayed responsiveness, dazed appearance, fatigue, pupil dilation, compromised vision, and difficulty breathing.

47. The method of any one of claims 24-45, wherein the one or more sequel are selected from concentration and memory deficiencies, irritability and/or other personality changes, sensitivity to light and/or sound, changes in sleep patterns, changes to ability to taste and/or smell, and psychological adjustment issues and depression.

48. A kit comprising
an oral formulation comprising an effective amount of a BBB-permeable epichaperome inhibitor, and
instructions for use to treat a traumatic brain injury.

49. The kit of claim 48, wherein the oral formulation is a solid form.

50. The kit of claim 49, wherein the oral formulation is a capsule, tablet, lozenge, or sublingual formulation.

51. The kit of claim 48, wherein the oral formulation is a liquid form.

52. The kit of claim 51, wherein the liquid form is an oral solution or an oral suspension.

53. A kit comprising
an intranasal or inhaled formulation comprising an effective amount of a BBB-permeable epichaperome inhibitor, and
instructions for use to treat a traumatic brain injury.

54. The kit of claim 53, wherein the intranasal or inhaled formulation is an intranasal formulation.

55. The kit of claim 54, wherein the intranasal formulation is a spray.

56. The kit of claim 53, wherein the intranasal or inhaled formulation is an inhaled formulation.

57. The kit of claim 56 further comprising an inhaler.

58. The kit of claim 56, further comprising a nebulizer.

59. A kit comprising
a parenteral formulation comprising an effective amount of a BBB-permeable epichaperome inhibitor, and
instructions for use to treat a traumatic brain injury.

60. The kit of claim 59, wherein the parenteral formulation is an injectable formulation.

61. The kit of claim 60, wherein the parenteral formulation is an intramuscular injectable formulation.

62. The kit of claim 60 or 61, wherein the parenteral formulation is provided in a syringe.

63. The kit of claim 61, wherein the parenteral formulation is provided in an auto-injector device or system.

64. The kit of any one of claims 48-63, further comprising a secondary therapeutic agent.

65. The kit of claim 64, wherein the secondary therapeutic agent is an anti-inflammatory agent.

66. The kit of claim 64, wherein the secondary therapeutic agent is an analgesic.

67. The kit of claim 64, wherein the secondary therapeutic agent is an anti-inflammatory agent and an analgesic.

68. The kit of any one of claims 64-67, wherein the kit comprises two or more BBB-permeable epichaperome inhibitors.

69. The kit of any one of claims 64-68, wherein the kit comprises a BBB-permeable epichaperome inhibitor and a BBB non-permeable epichaperome inhibitor.

70. The kit of any one of claims 64-67, wherein the kit comprises multiple doses of the BBB-permeable epichaperome inhibitor.

71. The kit of claim 70, wherein the kit comprises a counter.
